(19) United States
(12) Patent Application Publication
(10) Pub. No.: US 2003/0158109 A1
(43) Pub. Date: Aug. 21, 2003

(54) Metastatic breast and colon cancer regulated genes

(76) Inventor: Klaus Giese, Berlin (DE)

Correspondence Address: Chiron Corporation, Intellectual Property, R338, P.O. Box 8097, Emeryville, CA, 94662-8097, US

(21) Appl. No.: 10/236,006

(22) Filed: Sep. 5, 2002

Related U.S. Application Data
(62) Division of application No. 09/712,016, filed on Nov. 13, 2000, now Pat. No. 6,468,790.

(51) Int. Cl. C12Q 1/68; G01N 33/574; C07H 21/04; A61K 38/17; C12P 21/02; C12N 5/06; C07K 14/47; C12N 9/00

(52) U.S. Cl. 514/12; 435/6; 435/69.1; 435/183; 435/320.1; 435/7.23; 435/325; 530/350; 536/23.2

(57) ABSTRACT

Gene sequences as shown in SEQ ID NOS: 1-85 have been found to be significantly associated with metastatic potential of cancer cells, especially breast and colon cancer cells. Methods are provided for determining the risk of metastasis of a tumor, which involve determining whether a tissue sample from a tumor expresses a polypeptide encoded by a gene as shown in SEQ ID NOS: 1-85, or a substantial portion thereof.

### TECHNICAL FIELD OF THE INVENTION

This invention relates to methods for predicting the behavior of tumors. More particularly, the invention relates to methods in which a tumor sample is examined for expression of a specified gene sequence thereby to indicate propensity for metastatic spread.

### BACKGROUND OF THE INVENTION

Breast cancer is one of the most common malignant diseases in women, with about 1,000,000 new cases per year worldwide. Colon cancer is another of the most common cancers. Despite use of a number of histochemical, genetic, and immunological markers, clinicians still have a difficult time predicting which tumors will metastasize to other organs. Some patients are in need of adjuvant therapy to prevent recurrence and metastasis and others are not. However, distinguishing between these subpopulations of patients is not straightforward, and course of treatment is not easily charted. There is a need in the art for new markers for distinguishing between tumors which will or have metastasized and those which are less likely to metastasize

### SUMMARY OF THE INVENTION

It is an object of the present invention to provide markers for distinguishing between tumors which will or have metastasized and those which are less likely to metastasize. These and other objects of the invention are provided by one or more of the embodiments described below.

One embodiment of the invention provides an isolated and purified human protein having an amino acid sequence which is at least 85% identical to an amino acid sequence encoded by a nucleotide sequence selected from the group consisting of SEQ ID NOS: 1-63 or the complement thereof.

Another embodiment of the invention provides a fusion protein which comprises a first protein segment and a second protein segment fused to each other by means of a peptide bond. The first protein segment consists of at least six contiguous amino acids selected from an amino acid sequence encoded by a nucleotide sequence selected from the group consisting of SEQ ID NOS: 1-63 or the complement thereof.

Yet another embodiment of the invention provides an isolated and purified polypeptide consisting of at least six contiguous amino acids of a human protein having an amino acid sequence encoded by a nucleotide sequence selected from the group consisting of SEQ ID NOS: 1-63 or the complement thereof.

Still another embodiment of the invention provides a preparation of antibodies which specifically bind to a human protein which comprises an amino acid sequence encoded by a nucleotide sequence selected from the group consisting of SEQ ID NOS: 1-63 or the complement thereof.

Even another embodiment of the invention provides an isolated and purified subgenomic polynucleotide comprising at least 11 contiguous nucleotides of a nucleotide sequence which is at least 96% identical to a nucleotide sequence selected from the group consisting of SEQ ID NOS: 1-63 or the complement thereof.

Another embodiment of the invention provides an isolated and purified gene which comprises a coding sequence comprising a nucleotide sequence selected from the group consisting of SEQ ID NOS: 1-63 or the complement thereof.

Yet another embodiment of the invention provides a method for determining metastasis in a tissue sample. An expression product of a gene which comprises a coding sequence selected from the group consisting of SEQ ID NOS: 1, 2, 4, 5, 9, 11, 13, 14, 18, 19, 20, 22, 24, 26, 29, 30, 33, 35, 36, 38-41, 45, 48, 52, 55, 57, 58, 60, 63-66, 69-74, 76, 80, 82, and 83 is measured in a tissue sample. A tissue sample which expresses the product is categorized as metastatic.

Still another embodiment of the invention provides a method for determining metastasis in a tissue sample. An expression product of a gene which comprises a sequence selected from the group consisting of SEQ ID NOS: 3, 7, 8, 10, 12, 15-17, 21, 23, 28, 31, 34, 37, 42-44, 46, 47, 49-51, 53, 59, 61, 62, 67, 68, 75, 77-79, 81, 84, and 85 is measured in a tissue sample. A tissue sample which does not express the product is categorized as metastatic.

Even another embodiment of the invention provides a method for determining metastatic potential in a tissue sample. An expression product of a gene which comprises a sequence selected from the group consisting of SEQ ID NOS: 1, 2, 4, 5, 9, 11, 13, 14, 18, 19, 20, 22, 24, 26, 29, 30, 33, 35, 36, 38-41, 45, 48, 52, 55, 57, 58, 60, 63-66, 69-74, 76, 80, 82, and 83 is measured in a tissue sample. A tissue sample which expresses the product is categorized as having metastatic potential.

A further embodiment of the invention provides a method for determining metastatic potential in a tissue sample. An expression product of a gene which comprises a sequence selected from the group consisting of SEQ ID NOS: 3, 7, 8, 10, 12, 15-17, 21, 23, 28, 31, 34, 37, 42-44, 46, 47, 49-51, 53, 59, 61, 62, 67, 68, 75, 77-79, 81, 84, and 85 is measured in a tissue sample. A tissue sample which does not express the product is categorized as having metastatic potential.

Another embodiment of the invention provides a method of predicting the propensity for metastatic spread of a breast tumor preferentially to bone or lung. An expression product of a gene which comprises a sequence selected from the group consisting of SEQ ID NO: 1, 5, 11, 18, 20, 22, 24, 30, 33, 35, 36, 38, 45, 52, 58, 65, 66, 70, 74, 76, and 80 is measured in a breast tumor sample. A breast tumor sample which expresses the product is categorized as having a propensity to metastasize to bone or lung.

Even another embodiment of the invention provides a method of predicting propensity for metastatic spread of a breast tumor preferentially to lung. An expression product of a gene which comprises a sequence selected from the group consisting of SEQ ID NOS: 2, 4, 9, 13, 14, 19, 26, 29, 39-41, 48, 55, 57, 60, 63, 64, 72, 73, 82, and 83 is measured in a breast tumor sample. A breast tumor sample which expresses the product is characterized as having a propensity to metastasize to lung.

Still another embodiment of the invention provides a method of predicting propensity for metastatic spread of a colon tumor. An expression product of a gene which comprises the nucleotide sequence shown in SEQ ID NO: 56 is measured in a colon tumor sample. A colon tumor sample which expresses the product is characterized as having a low propensity to metastasize.

Even another embodiment of the invention provides a method for determining metastasis in a tissue sample. An expression product of a gene which comprises a coding sequence selected from the group consisting of SEQ ID NOS: 3, 7, 8, 10, 12, 15-17, 21, 23, 25, 28, 31, 34, 37, 42-44, 46, 47, 49, 61, 62, 67, 68, 75, 77-79, 81, 84, and 85 is measured in a tissue sample. A tissue sample which expresses the product is categorized as non-metastatic.

Yet another embodiment of the invention provides a method for determining metastasis in a tissue sample. An expression product of a gene which comprises a coding sequence selected from the group consisting of SEQ ID NOS: 3, 7, 8, 10, 12, 15-17, 21, 23, 25, 28, 31, 34, 37, 42-44, 46, 47, 49, 61, 62, 67, 68, 75, 77-79, 81, 84, and 85 is measured in a tissue sample. A tissue sample which does not express the product is categorized as metastatic.

The invention thus provides the art with a number of genes and proteins, which can be used as markers of metastasis. These are useful for more rationally prescribing the course of therapy for breast or colon cancer patients.

### DETAILED DESCRIPTION

It is a discovery of the present invention that a number of genes are differentially expressed between metastatic cancer cells, especially cancer cells of the breast and colon, and non-metastatic cancer cells. These genes are metastatic marker genes. This information can be utilized to make diagnostic reagents specific for the expression products of the differentially expressed genes. It can also be used in diagnostic and prognostic methods which will help clinicians in planning appropriate treatment regimes for cancers, especially of the breast or colon.

Some of the polynucleotides disclosed herein represent novel genes which are differentially expressed between non-metastatic cancer cells and cancer cells which have a potential to metastasize. SEQ ID NOS: 1-63 represent novel metastatic marker genes (Table 1). SEQ ID NOS: 64-85 represent known genes which have been found to be differentially expressed in metastatic relative to non-metastatic cancer cells (Table 2). Some of the metastatic marker genes disclosed herein are expressed in metastatic cells relative to non-metastatic cells, particularly in breast cancer cells which metastasize to bone and lung (SEQ ID NOS: 1, 5, 11, 18, 20, 22, 24, 30, 33, 35, 36, 38, 45, 52, 58, 65, 66, 70, 74, 76, and 80). One metastatic marker gene (SEQ ID NO: 56) is expressed in non-metastatic breast cancer cells and in colon cancer cells with low metastatic potential. Other metastatic marker genes are expressed in metastatic cancer cells, particularly in breast cancer cells which metastasize only to lung (SEQ ID NOS: 2, 4, 9, 13, 14, 19, 26, 29, 39-41, 48, 55, 57, 60, 63, 64, 72, 73, 82, and 83). Still other metastatic marker genes (SEQ ID NOS: 3, 7, 8, 10, 12, 15-17, 21, 23, 28, 31, 34, 37, 42-44, 46, 47, 49, 61, 62, 67, 68, 75, 77-79, 81, 84, and 85) are expressed in cancer cells which do not typically metastasize, particularly in breast cancer cells. Identification of these relationships and markers permits the formulation of reagents and methods as further described below. Other metastatic marker genes, such as those which comprise a nucleotide sequence shown in SEQ ID NOS: 6, 27, 32, and 54, can be used to identify cancerous tissue, particularly breast cancer tissue.

Sequences of metastatic marker genes are disclosed in SEQ ID NOS: 1-85. Metastatic marker proteins can be made by expression of the disclosed polynucleotide molecules. Amino acid sequences encoded by novel polynucleotides of the invention can be predicted by running a translation program for each of three reading frames for a disclosed sequence and its complement. Complete polynucleotide sequences can be obtained by chromosome walking, screening of libraries for overlapping clones, 5′ RACE, or other techniques well known in the art.

Reference to metastatic marker nucleotide or amino acid sequences includes variants which have similar expression patterns in metastatic relative to non-metastatic cells, as described below. Metastatic marker polypeptides can differ in length from full-length metastatic marker proteins and contain at least 6, 8, 10, 12, 15, 18, 20, 25, 30, 35, 40, 45, 50, 55, 60, 65, 70, 75, 80, 85, 90, 95, 100, 120, 140, 160, 180, or 200 or more contiguous amino acids of a metastatic marker protein.

Variants of marker proteins and polypeptides can also occur. Metastatic marker protein or polypeptide variants can be naturally or non-naturally occurring. Naturally occurring metastatic marker protein or polypeptide variants are found in humans or other species and comprise amino acid sequences which are substantially identical to the proteins encoded by genes corresponding to the nucleotide sequences shown in SEQ ID NOS: 1-85 or their complements. Non-naturally occurring metastatic marker protein or polypeptide variants which retain substantially the same differential expression patterns in metastatic relative to non-metastatic cancer cells as naturally occurring metastatic marker protein or polypeptide variants are also included here. Preferably, naturally or non-naturally occurring metastatic marker protein or polypeptide variants have amino acid sequences which are at least 85%, 90%, or 95% identical to amino acid sequences encoded by the nucleotide sequences shown in SEQ ID NOS: 1-85. More preferably, the molecules are at least 98% or 99% identical. Percent sequence identity between a wild-type protein or polypeptide and a variant is determined by aligning the wild-type protein or polypeptide with the variant to obtain the greatest number of amino acid matches, as is known in the art, counting the number of amino acid matches between the wild-type and the variant, and dividing the total number of matches by the total number of amino acid residues of the wild-type sequence.

Preferably, amino acid changes in metastatic marker protein or polypeptide variants are conservative amino acid changes, i.e., substitutions of similarly charged or uncharged amino acids. A conservative amino acid change involves substitution of one of a family of amino acids which are related in their side chains. Naturally occurring amino acids are generally divided into four families: acidic (aspartate, glutamate), basic (lysine, arginine, histidine), non-polar (alanine, valine, leucine, isoleucine, proline, phenylalanine, methionine, tryptophan), and uncharged polar (glycine, asparagine, glutamine, cystine, serine, threonine, tyrosine) amino acids. Phenylalanine, tryptophan, and tyrosine are sometimes classified jointly as aromatic amino acids.

It is reasonable to expect that an isolated replacement of a leucine with an isoleucine or valine, an aspartate with a glutamate, a threonine with a serine, or a similar replacement of an amino acid with a structurally related amino acid will not have a major effect on the biological properties of the resulting metastatic marker protein or polypeptide variant. Properties and functions of metastatic marker protein or polypeptide variants are of the same type as a metastatic marker protein or polypeptide comprising amino acid sequences encoded by the nucleotide sequences shown in SEQ ID NOS: 1-85, although the properties and functions of variants can differ in degree. Whether an amino acid change results in a metastatic marker protein or polypeptide variant with the appropriate differential expression pattern can readily be determined. For example, nucleotide probes can be selected from the marker gene sequences disclosed herein and used to detect marker gene mRNA in Northern blots or in tissue sections, as is known in the art. Alternatively, antibodies which specifically bind to protein products of metastatic marker genes can be used to detect expression of metastatic marker proteins.

Metastatic marker variants include glycosylated forms, aggregative conjugates with other molecules, and covalent conjugates with unrelated chemical moieties. Metastatic marker variants also include allelic variants, species variants, and muteins. Truncations or deletions of regions which do not affect the differential expression of metastatic marker genes are also metastatic marker variants. Covalent variants can be prepared by linking functionalities to groups which are found in the amino acid chain or at the N- or C-terminal residue, as is known in the art.

Full-length metastatic marker proteins can be extracted, using standard biochemical methods, from metastatic marker protein-producing human cells, such as metastatic breast or colon cancer cells. An isolated and purified metastatic marker protein or polypeptide is separated from other compounds which normally associate with a metastatic marker protein or polypeptide in a cell, such as certain proteins, carbohydrates, lipids, or subcellular organelles. A preparation of isolated and purified metastatic marker proteins or polypeptides is at least 80% pure; preferably, the preparations are 90%, 95%, or 99% pure.

Metastatic marker proteins and polypeptides can also be produced by recombinant DNA methods or by synthetic chemical methods. For production of recombinant metastatic marker proteins or polypeptides, coding sequences selected from the nucleotide sequences shown in SEQ ID NOS: 1-85, or variants of those sequences which encode metastatic marker proteins, can be expressed in known prokaryotic or eukaryotic expression systems (see below). Bacterial, yeast, insect, or mammalian expression systems can be used, as is known in the art.

Alternatively, synthetic chemical methods, such as solid phase peptide synthesis, can be used to synthesize a metastatic marker protein or polypeptide. General means for the production of peptides, analogs or derivatives are outlined in CHEMISTRY AND BIOCHEMISTRY OF AMINO ACIDS, PEPTIDES, AND PROTEINS—A SURVEY OF RECENT DEVELOPMENTS, Weinstein, B. ed., Marcell Dekker, Inc., publ., New York (1983). Moreover, substitution of D-amino acids for the normal L-stereoisomer can be carried out to increase the half-life of the molecule. Metastatic marker variants can be similarly produced.

Non-naturally occurring fusion proteins comprising at least 6, 8, 10, 12, 15, 18, 20, 25, 30, 35, 40, 50, 55, 60. 65, 70, 75, 80, 85, 90, 95, 100, 120, 140, 160, 180, or 200 or more contiguous metastatic marker amino acids can also be constructed. Human metastatic marker fusion proteins are useful for generating antibodies against metastatic marker amino acid sequences and for use in various assay systems. For example, metastatic marker fusion proteins can be used to identify proteins which interact with metastatic marker proteins and influence their functions Physical methods, such as protein affinity chromatography, or library-based assays for protein-protein interactions, such as the yeast two-hybrid or phage display systems, can also be used for this purpose. Such methods are well known in the art and can also be used as drug screens.

A metastatic marker fusion protein comprises two protein segments fused together by means of a peptide bond. The first protein segment comprises at least 6, 8, 10, 12, 15, 18, 20, 25, 30, 35, 40, 50, 55, 60, 65, 70, 75, 80, 85, 90, 95, 100, 120, 140, 160, 180, or 200 or more contiguous amino acids of a metastatic marker protein. The amino acids can be selected from the amino acid sequences encoded by the nucleotide sequences shown in SEQ ID NOS: 1-85 or from variants of those sequences, such as those described above. The first protein segment can also comprise a full-length metastatic marker protein.

The second protein segment can be a full-length protein or a protein fragment or polypeptide. The fusion protein can be labeled with a detectable marker, as is known in the art, such as a radioactive, fluorescent, chemiluminescent, or biotinylated marker. The second protein segment can be an enzyme which will generate a detectable product, such as β-galactosidase. The first protein segment can be N-terminal or C-terminal, as is convenient.

Techniques for making fusion proteins, either recombinantly or by covalently linking two protein segments, are also well known. Recombinant DNA methods can be used to prepare metastatic marker fusion proteins, for example, by making a DNA construct which comprises coding sequences selected from SEQ ID NOS: 1-85 in proper reading frame with nucleotides encoding the second protein segment and expressing the DNA construct in a host cell, as described below.

Isolated and purified metastatic marker proteins, polypeptides, variants, or fusion proteins can be used as immunogens, to obtain preparations of antibodies which specifically bind to a metastatic marker protein. The antibodies can be used, inter alia, to detect wild-type metastatic marker proteins in human tissue and fractions thereof. The antibodies can also be used to detect the presence of mutations in metastatic marker genes which result in under- or over-expression of a metastatic marker protein or in expression of a metastatic marker protein with altered size or electrophoretic mobility.

Preparations of polyclonal or monoclonal antibodies can be made using standard methods. Single-chain antibodies can also be prepared. Single-chain antibodies which specifically bind to metastatic marker proteins, polypeptides, variants, or fusion proteins can be isolated, for example, from single-chain immunoglobulin display libraries, as is known in the art. The library is “panned” against metastatic marker protein amino acid sequences, and a number of single chain antibodies which bind with high-affinity to different epitopes of metastatic marker proteins can be isolated. Hayashi et al., 1995, Gene 160:129-30. Single-chain antibodies can also be constructed using a DNA amplification method, such as the polymerase chain reaction (PCR), using hybridoma cDNA as a template. Thirion et al., 1996, Eur. J. Cancer Prev. 5:507-11.

Single-chain antibodies can be mono- or bispecific, and can be bivalent or tetravalent. Construction of tetravalent, bispecific single-chain antibodies is taught in Coloma and Morrison, 1997, Nat. Biotechnol. 15:159-63. Construction of bivalent, bispecific single-chain antibodies is taught in Mallender and Voss, 1994, J. Biol. Chem. 269:199-206.

A nucleotide sequence encoding the single-chain antibody can be constructed using manual or automated nucleotide synthesis, cloned into DNA expression constructs using standard recombinant DNA methods, and introduced into cells which express the coding sequence, as described below. Alternatively, single-chain antibodies can be produced directly using, for example, filamentous phage technology. Verhaar et al., 1995, Int. J. Cancer 61:497-501; Nicholls et al., 1993, J. Immunol. Meth. 165:81-91.

Metastatic marker-specific antibodies specifically bind to epitopes present in a full-length metastatic marker protein having an amino acid sequence encoded by a nucleotide sequence shown in SEQ ID NOS: 1-85, to metastatic marker polypeptides, or to metastatic marker variants, either alone or as part of a fusion protein. Preferably, metastatic marker epitopes are not present in other human proteins. Typically, at least 6, 8, 10, or 12 contiguous amino acids are required to form an epitope. However, epitopes which involve non-contiguous amino acids may require more, e.g., at least 15, 25, or 50 amino acids.

Antibodies which specifically bind to metastatic marker proteins, polypeptides, fusion proteins, or variants provide a detection signal at least 5-, 10-, or 20-fold higher than a detection signal provided with other proteins when used in Western blots or other immunochemical assays. Preferably, antibodies which specifically bind to metastatic marker epitopes do not detect other proteins in immunochemical assays and can immunoprecipitate a metastatic marker protein, polypeptide, fusion protein, or variant from solution.

Antibodies can be purified by methods well known in the art. Preferably, the antibodies are affinity purified, by passing the antibodies over a column to which a metastatic marker protein, polypeptide, variant, or fusion protein is bound. The bound antibodies can then be eluted from the column, for example, using a buffer with a high salt concentration.

Subgenomic polynucleotides contain less than a whole chromosome. Preferably, the polynucleotides are intron-free. In a preferred embodiment, the polynucleotide molecules comprise a contiguous sequence of 10, 11, 12, 15, 20, 25, 30, 32, 35, 40, 45, 50, 60, 70, 74, 80, 90, 100, 125, 150, 154, 175, 182, 200, 243, or 268 nucleotides selected from SEQ ID NOS: 1-85 or the complements thereof. The complement of a nucleotide sequence shown in SEQ ID NOS: 1-85 is a contiguous nucleotide sequence which forms Watson-Crick base pairs with a contiguous nucleotide sequence shown in SEQ ID NOS: 1-85. The complement of a nucleotide sequence shown in SEQ ID NOS: 1-85 (the antisense strand) is also a subgenomic polynucleotide, and can be used provide marker protein antisense oligonucleotides. Double-stranded polynucleotides which comprise one of the nucleotide sequences shown in SEQ ID NOS: 1-85 are also subgenomic polynucleotides. Metastatic marker protein subgenomic polynucleotides also include polynucleotides which encode metastatic marker protein-specific single-chain antibodies and ribozymes, or fusion proteins comprising metastatic marker protein amino acid sequences.

Degenerate nucleotide sequences encoding amino acid sequences of metastatic marker protein and or variants, as well as homologous nucleotide sequences which are at least 85%, 90%, -95%, 98%, or 99% identical to the nucleotide sequences shown in SEQ ID NOS: 1-85, are also metastatic marker subgenomic polynucleotides. Typically, homologous metastatic marker subgenomic polynucleotide sequences can be confirmed by hybridization under stringent conditions, as is known in the art. Percent sequence identity between wild-type and homologous variant sequences is determined by aligning the wild-type polynucleotide with the variant to obtain the greatest number of nucleotide matches, as is known in the art, counting the number of nucleotide matches between the wild-type and the variant, and dividing the total number of matches by the total number of nucleotides of the wild-type sequence. A preferred algorithm for calculating percent identity is the Smith-Waterman homology search algorithm as implemented in MPSRCH program (Oxford Molecular) using an affine gap search with the following search parameters: gap open penalty of 10, and gap extension penalty of 1.

Metastatic marker subgenomic polynucleotides can be isolated and purified free from other nucleotide sequences using standard nucleic acid purification techniques. For example, restriction enzymes and probes can be used to isolate polynucleotide fragments which comprise nucleotide sequences encoding a metastatic marker protein. Isolated and purified subgenomic polynucleotides are in preparations which are free or at least 90% free of other molecules.

Complementary DNA molecules which encode metastatic marker proteins can be made using reverse transcriptase, with metastatic marker mRNA as a template. The polymerase chain reaction (PCR) or other amplification techniques can be used to obtain metastatic marker subgenomic polynucleotides, using either human genomic DNA or cDNA as a template, as is known in the art. Alternatively, synthetic chemistry techniques can be used to synthesize metastatic marker subgenomic polynucleotides which comprise coding sequences for regions of metastatic marker proteins, single-chain antibodies, or ribozymes, or which comprise antisense oligonucleotides. The degeneracy of the genetic code allows alternate nucleotide sequences to be synthesized which will encode a metastatic marker protein comprising amino acid sequences encoded by the nucleotide sequences shown in SEQ ID NOS: 1-85.

Purified and isolated metastatic marker subgenomic polynucleotides can be used as primers to obtain additional copies of the polynucleotides or as probes for identifying wild-type and mutant metastatic marker protein coding sequences. Metastatic marker subgenomic polynucleotides can be used to express metastatic marker mRNA, protein, polypeptides, or fusion proteins and to generate metastatic marker antisense oligonucleotides and ribozymes.

A metastatic marker subgenomic polynucleotide comprising metastatic marker protein coding sequences can be used in an expression construct. Preferably, the metastatic marker subgenomic polynucleotide is inserted into an expression plasmid (for example, the Ecdyson system, pIND, In Vitro Gene). Metastatic marker subgenomic polynucleotides can be propagated in vectors and cell lines using techniques well known in the art. Metastatic marker subgenomic polynucleotides can be on linear or circular molecules. They can be on autonomously replicating molecules or on molecules without replication sequences. They can be regulated by their own or by other regulatory sequences, as are known in the art.

A host cell comprising a metastatic marker expression construct can then be used to express all or a portion of a metastatic marker protein. Host cells comprising metastatic marker expression constructs can be prokaryotic or eukaryotic. A variety of host cells are available for use in bacterial, yeast, insect, and human expression systems and can be used to express or to propagate metastatic marker expression constructs (see below). Expression constructs can be introduced into host cells using any technique known in the art. These techniques include transferrin-polycation-mediated DNA transfer, transfection with naked or encapsulated nucleic acids, liposome-mediated cellular fusion, intracellular transportation of DNA-coated latex beads, protoplast fusion, viral infection, electroporation, and calcium phosphate-mediated transfection.

A metastatic marker expression construct comprises a promoter which is functional in a chosen host cell. The skilled artisan can readily select an appropriate promoter from the large number of cell type-specific promoters known and used in the art. The expression construct can also contain a transcription terminator which is functional in the host cell. The expression construct comprises a polynucleotide segment which encodes all or a portion of the metastatic marker protein, variant, fusion protein, antibody, or ribozyme. The polynucleotide segment is located downstream from the promoter. Transcription of the polynucleotide segment initiates at the promoter. The expression construct can be linear or circular and can contain sequences, if desired, for autonomous replication.

Bacterial systems for expressing metastatic marker expression constructs include those described in Chang et al., Nature (1978) 275: 615, Goeddel et al., Nature (1979) 281: 544, Goeddel et al., Nucleic Acids Res. (1980) 8: 4057, EP 36,776, U.S. Pat. No. 4,551,433, deBoer et al., Proc. Nat'l Acad Sci. USA (1983) 80: 21-25, and Siebenlist et al., Cell (1980) 20: 269.

Expression systems in yeast include those described in Hinnen et al., Proc. Nat'l Acad. Sci. USA (1978) 75: 1929; Ito et al, J. Bacteriol. (1983) 153: 163; Kurtz et al., Mol. Cell. Biol. (1986) 6: 142; Kunze et al., J. Basic Microbiol. (1985) 25: 141; Gleeson et al., J. Gen. Microbiol. (1986) 132: 3459, Roggenkamp et al., Mol. Gen. Genet. (1986) 202 :302) Das et al., J. Bacteriol. (1984) 158: 1165; De Louvencourt et al., J. Bacteriol. (1983) 154: 737, Van den Berg et al., Bio/Technology (1990) 8: 135; Kunze et al., J. Basic Microbiol. (1985) 25: 141; Cregg et al., Mol, Cell. Biol. (1985) 5: 3376, U.S. Pat. No. 4,837,148, U.S. Pat. No. 4,929,555; Beach and Nurse, Nature (1981) 300: 706; Davidow et al., Curr. Genet. (1985) 10: 380, Gaillardin et al., Curr. Genet. (1985) 10: 49, Ballance et al., Biochem. Biophys. Res. Commun. (1983) 112: 284-289; Tilburn et al., Gene (1983) 26: 205-221, Yelton et al., Proc. Nat'l Acad. Sci USA (1984) 81: 1470-1474, Kelly and Hynes, EMBO J. (1985) 4: 475479; EP 244,234, and WO 91/00357.

Expression of metastatic marker expression constructs in insects can be carried out as described in U.S. Pat. No. 4,745,051, Friesen et al. (1986) “The Regulation of Baculovirus Gene Expression” in: THE MOLECULAR BIOLOGY OF BACULOVIRUSES (W. Doerfler, ed.), EP 127,839, EP 155,476, and Vlak et al., J. Gen. Virol. (1988) 69: 765-776, Miller et al., Ann. Rev. Microbiol. (1988) 42: 177, Carbonell et al., Gene (1988) 73: 409, Maeda et al., Nature (1985) 315: 592-594, Lebacq-Verheyden et al., Mol. Cell. Biol. (1988) 8: 3129; Smith et al., Proc. Nat'l Acad Sci. USA (1985) 82: 8404, Miyajima et al., Gene (1987) 58: 273; and Martin et al., DNA (1988) 7:99. Numerous baculoviral strains and variants and corresponding permissive insect host cells from hosts are described in Luckow et al., Bio/Technology (1988) 6: 47-55, Miller et al., in GENETIC ENGINEERING (Setlow, J. K. et al. eds.), Vol. 8 (Plenum Publishing, 1986), pp. 277-279, and Maeda et al., Nature, (1985) 315: 592-594.

Mammalian expression of metastatic marker expression constructs can be achieved as described in Dijkema et al., EMBO J. (1985) 4: 761, Gorman et al., Proc. Nat'l Acad. Sci. USA (1982b) 79: 6777, Boshart et al., Cell (1985) 41: 521 and U.S. Pat No. 4,399,216. Other features of mammalian expression of metastatic marker expression constructs can be facilitated as described in Ham and Wallace, Meth. Enz. (1979) 58: 44, Barnes and Sato, Anal. Biochem. (1980) 102: 255, U.S. Pat. No. 4,767,704, U.S. Pat. No. 4,657,866. U.S. Pat. No. 4,927,762, U.S. Pat. No. 4,560,655, WO 90/103430, WO 87/00195, and U.S. Pat. No. RE 30,985.

Subgenomic polynucleotides of the invention can also be used in gene delivery vehicles, for the purpose of delivering a metastatic marker mRNA or oligonucleotide (either with the sequence of native metastatic marker mRNA or its complement), full-length metastatic marker protein, metastatic marker fusion protein, metastatic marker polypeptide, or metastatic marker-specific ribozyme or single-chain antibody, into a cell preferably a eukaryotic cell. According to the present invention, a gene delivery vehicle can be, for example, naked plasmid DNA, a viral expression vector comprising a metastatic marker subgenomic polynucleotide, or a metastatic marker subgenomic polynucleotide in conjunction with a liposome or a condensing agent.

In one embodiment of the invention, the gene delivery vehicle comprises a promoter and a metastatic marker subgenomic polynucleotide. Preferred promoters are tissue-specific promoters and promoters which are activated by cellular proliferation, such as the thymidine kinase and thymidylate synthase promoters. Other preferred promoters include promoters which are activatable by infection with a virus, such as the α- and β-interferon promoters, and promoters which are activatable by a hormone, such as estrogen. Other promoters which can be used include the Moloney virus LTR, the CMV promoter, and the mouse albumin promoter.

A metastatic marker gene delivery vehicle can comprise viral sequences such as a viral origin of replication or packaging signal. These viral sequences can be selected from viruses such as astrovirus, coronavirus, orthomyxovirus, papovavirus, paramyxovirus, parvovirus, picomavirus, poxvirus, retrovirus, togavirus or adenovirus. In a preferred embodiment, the metastatic marker gene delivery vehicle is a recombinant retroviral vector. Recombinant retroviruses and various uses thereof have been described in numerous references including, for example, Mann et al., Cell 33:153, 1983, Cane and Mulligan, Proc. Nat'l Acad. Sci. USA 81:6349, 1984, Miller et al., Human Gene Therapy 1:5-14, 1990, U.S. Pat. Nos. 4,405,712, 4,861,719, and 4,980,289, and PCT Application Nos. WO 89/02,468, WO 89/05,349, and WO 90/02,806. Numerous retroviral gene delivery vehicles can be utilized in the present invention, including for example those described in EP 0,415,731; WO 90/07936; WO 94/03622; WO 93/25698; WO 93/25234; U.S. Pat. No. 5,219,740; WO 9311230; WO 9310218; Vile and Hart, Cancer Res. 53:3860-3864, 1993; Vile and Hart, Cancer Res. 53:962-967, 1993; Ram et al., Cancer Res. 53:83-88, 1993; Takamiya et al., J. Neurosci. Res. 33:493-503, 1992; Baba et al., J. Neurosurg. 79:729-735, 1993 (U.S. Pat. No. 4,777,127, GB 2,200,651, EP 0,345,242 and WO91/02805).

Particularly preferred retroviruses are derived from retroviruses which include avian leukosis virus (ATCC Nos. VR-535 and VR-247), bovine leukemia virus (VR-1315), murine leukemia virus (MLV), mink-cell focus-inducing virus (Koch et al., J. Vir. 49:828, 1984; and Oliff et al., J. Vir. 48:542, 1983), murine sarcoma virus (ATCC Nos. VR-844, 45010 and 45016), reticuloendotheliosis virus (ATCC Nos VR-994, VR-770 and 45011), Rous sarcoma virus, Mason-Pfizer monkey virus, baboon endogenous virus, endogenous feline retrovirus (e.g.. RD114), and mouse or rat gL30 sequences used as a retroviral vector. Particularly preferred strains of MLV from which recombinant retroviruses can be generated include 4070A and 1504A (Hartley and Rowe, J. Vir. 19:19, 1976), Abelson (ATCC No. VR-999), Friend (ATCC No. VR-245), Graffi (Ru et al., J. Vir. 67:4722, 1993; and Yantchev Neoplasma 26:397, 1979), Gross (ATCC No. VR-590), Kirsten (Albino et al., J. Exp. Med. 164:1710, 1986), Harvey sarcoma virus (Manly et al., J. Vir. 62:3540, 1988; and Albino et al., J. Exp. Med. 164:1710, 1986) and Rauscher (ATCC No. VR-998), and Moloney MLV (ATCC No. VR-190). A particularly preferred non-mouse retrovirus is Rous sarcoma virus. Preferred Rous sarcoma viruses include Bratislava (Manly et al., J. Vir. 62:3540, 1988; and Albino et al., J. Exp. Med. 164:1710, 1986), Bryan high titer (e.g., ATCC Nos. VR-334, VR-657, VR-726, VR-659, and VR-728), Bryan standard (ATCC No. VR-140), Carr-Zilber (Adgighitov et al., Neoplasma 27:159, 1980), Engelbreth-Holm (Laurent et al., Biochem Biophys Acta 908:241, 1987), Harris, Prague (e.g., ATCC Nos. VR-772, and 45033), and Schmidt-Ruppin (e.g., ATCC Nos. VR-724, VR-725, VR-354) viruses.

Any of the above retroviruses can be readily utilized in order to assemble or construct retroviral metastatic marker gene delivery vehicles given the disclosure provided herein and standard recombinant techniques (e.g., Sambrook et al., Molecular Cloning: A Laboratory Manual, 2d ed., Cold Spring Harbor Laboratory Press, 1989, and Kunkle, PNAS 82:488, 1985) known in the art. Portions of retroviral Metastatic marker expression vectors can be derived from different retroviruses. For example, retrovector LTRs can be derived from a murine sarcoma virus, a tRNA binding site from a Rous sarcoma virus, a packaging signal from a murine leukemia virus, and an origin of second strand synthesis from an avian leukosis virus. These recombinant retroviral vectors can be used to generate transduction competent retroviral vector particles by introducing them into appropriate packaging cell lines (see Ser. No. 07/800,921, filed Nov. 29, 1991). Recombinant retroviruses can be produced which direct the site-specific integration of the recombinant retroviral genome into specific regions of the host cell DNA. Such site-specific integration can be mediated by a chimeric integrase incorporated into-the retroviral particle (see Ser. No. 08/445.466 filed May 22, 1995). It is preferable that the recombinant viral gene delivery vehicle is a replication-defective recombinant virus.

Packaging cell lines suitable for use with the above-described retroviral gene delivery vehicles can be readily prepared (see Ser. No. 08/240,030, filed May 9, 1994; see also WO 92/05266) and used to create producer cell lines (also termed vector cell lines or “VCLs”) for production of recombinant viral particles. In particularly preferred embodiments of the present invention, packaging cell lines are made from human (e.g., HT1080 cells) or mink parent cell lines, thereby allowing production of recombinant retroviral gene delivery vehicles which are capable of surviving inactivation in human serum. The construction of recombinant retroviral gene delivery vehicles is described in detail in WO 91/02805. These recombinant retroviral gene delivery vehicles can be used to generate transduction competent retroviral particles by introducing them into appropriate packaging cell lines (see Ser. No. 07/800,921). Similarly, adenovirus gene delivery vehicles can also be readily prepared and utilized given the disclosure provided herein (see also Berkner, Biotechniques 6:616-627, 1988, and Rosenfeld et al., Science 252:431-434, 1991, WO 93/07283, WO 93/06223, and WO 93/07282).

A metastatic marker gene delivery vehicle can also be a recombinant adenoviral gene delivery vehicle. Such vehicles can be readily prepared and utilized given the disclosure provided herein (see Berkner, Biotechniques 6:616, 1988, and Rosenfeld et al., Science 252:431, 1991, WO 93/07283, WO 93/06223, and WO 93/07282). Adeno-associated viral metastatic marker gene delivery vehicles can also be constructed and used to deliver metastatic marker amino acids or nucleotides. The use of adeno-associated viral gene delivery vehicles in vitro is described in Chatterjee et al., Science 258: 1485-1488 (1992). Walsh et al., Proc. Nat'l Acad. Sci. 89: 7257-7261 (1992), Walsh et al., J. Clin. Invest. 94: 1440-1448 (1994), Flotte et al., J. Biol. Chem. 268: 3781-3790 (1993). Ponnazhagan et al., J. Exp. Med. 179: 733-738 (1994), Miller et al., Proc. Nat'l Acad. Sci. 91: 10183-10187 (1994), Einerhand et al., Gene Ther. 2: 336-343 (1995), Luo et al., Exp. Hematol. 23: 1261-1267 (1995), and Zhou et al., Gene Therapy 3: 223-229 (1996). In vivo use of these vehicles is described in Flotte et al., Proc. Nat'l Acad. Sci. 90: 10613-10617 (1993), and Kaplitt et al., Nature Genet. 8:148-153 (1994).

In another embodiment of the invention, a metastatic marker gene delivery vehicle is derived from a togavirus. Preferred togaviruses include alphaviruses, in particular those described in U.S. Ser. No. 08/405,627, filed Mar. 15, 1995, WO 95/07994. Alpha viruses, including Sindbis and ELVS viruses can be gene delivery vehicles for metastatic marker polynucleotides. Alpha viruses are described in WO 94/21792, WO 92/10578 and WO 95/07994. Several different alphavirus gene delivery vehicle systems can be constructed and used to deliver metastatic marker subgenomic polynucleotides to a cell according to the present invention. Representative examples of such systems include those described in U.S. Pat. Nos. 5,091,309 and 5,217,879. Particularly preferred alphavirus gene delivery vehicles for use in the present invention include those which are described in WO 95/07994, and U.S. Ser. No. 08/405,627.

Preferably, the recombinant viral vehicle is a recombinant alphavirus viral vehicle based on a Sindbis virus. Sindbis constructs, as well as numerous similar constructs, can be readily prepared essentially as described in U.S. Ser. No. 08/198,450. Sindbis viral gene delivery vehicles typically comprise a 5′ sequence capable of initiating Sindbis virus transcription, a nucleotide sequence encoding Sindbis non-structural proteins, a viral junction region inactivated so as to prevent subgenomic fragment transcription, and a Sindbis RNA polymerase recognition sequence. Optionally, the viral junction region can be modified so that subgenomic polynucleotide transcription is reduced, increased, or maintained. As will be appreciated by those in the art, corresponding regions from other alphaviruses can be used in place of those described above.

The viral junction region of an alphavirus-derived gene delivery vehicle can comprise a first viral junction region which has been inactivated in order to prevent transcription of the subgenomic polynucleotide and a second viral junction region which has been modified such that subgenomic polynucleotide transcription is reduced. An alphavirus-derived vehicle can also include a 5′ promoter capable of initiating synthesis of viral RNA from cDNA and a 3′ sequence which controls transcription termination.

Other recombinant togaviral gene delivery vehicles which can be utilized in the present invention include those derived from Semliki Forest virus (ATCC VR-67; ATCC VR-1247), Middleberg virus (ATCC VR-370), Ross River virus (ATCC VR-373; ATCC VR-1246), Venezuelan equine encephalitis virus (ATCC VR923; ATCC VR-1250; ATCC VR-1249; ATCC VR-532), and those described in U.S. Pat. Nos. 5,091,309 and 5,217,879 and in WO 92/10578. The Sindbis vehicles described above, as well as numerous similar constructs, can be readily prepared essentially as described in U.S. Ser. No. 08/198,450.

Other viral gene delivery vehicles suitable for use in the present invention include, for example, those derived from poliovirus (Evans et al., Nature 339:385, 1989, and Sabin et al., J. Biol Standardization 1:115, 1973) (ATCC VR-58); rhinovirus (Arnold et al., J. Cell. Biochem. L401, 1990) (ATCC VR-1110); pox viruses, such as canary pox virus or vaccinia virus (Fisher-Hoch et al., PNAS 86:317, 1989; Flexner et al., Ann. N.Y Acad. Sci. 569:86, 1989; Flexner et al., Vaccine 8:17, 1990; U.S. Pat. No. 4,603,112 and U.S. Pat. No. 4,769,330; WO 89/01973) (ATCC VR-111; ATCC VR-2010); SV40 (Mulligan et al., Nature 277:108, 1979) (ATCC VR-305), (Madzak et al., J. Gen. Vir. 73:1533, 1992); influenza virus (Luytjes et al., Cell 59:1107, 1989; McMicheal et al., The New England Journal of Medicine 309:13, 1983; and Yap et al., Nature 273:238, 1978) (ATCC VR-797); parvovirus such as adeno-associated virus (Samulski et al., J. Vir. 63:3822, 1989, and Mendelson et al., Virology 166:154, 1988) (ATCC VR-645); herpes simplex virus (Kit et al., Adv. Exp. Med. Biol. 215:219, 1989) (ATCC VR-977; ATCC VR-260); Nature 277: 108, 1979); human immunodeficiency virus (EPO 386,882, Buchschacher et al., J. Vir. 66:2731, 1992); measles virus (EPO 440,219) (ATCC VR-24); A (ATCC VR-67; ATCC VR-1247), Aura (ATCC VR368), Bebaru virus (ATCC VR-600; ATCC VR-1240), Cabassou (ATCC VR-922), Chikungunya virus (ATCC VR-64; ATCC VR-1241), Fort Morgan (ATCC VR-924), Getah virus (ATCC VR-369; ATCC VR-1243), Kyzylagach (ATCC VR-927), Mayaro (ATCC VR-66), Mucambo virus (ATCC VR-580; ATCC VR-1244), Ndumu (ATCC VR-371), Pixuna virus (ATCC VR-372; ATCC VR-1245), Tonate (ATCC VR-925), Triniti (ATCC VR-469), Una (ATCC VR-374), Whataroa (ATCC VR-926), Y-62-33 (ATCC VR-375), O'Nyong virus, Eastern encephalitis virus (ATCC VR-65; ATCC VR-1242), Western encephalitis virus (ATCC VR-70; ATCC VR-1251; ATCC VR-622; ATCC VR-1252), and coronavirus (Hamre et al., Proc. Soc. Exp. Biol. Med. 121:190, 1966) (ATCC VR-740).

A subgenomic metastatic marker polynucleotide of the invention can also be combined with a condensing agent to form a gene delivery vehicle. In a preferred embodiment, the condensing agent is a polycation, such as polylysine, polyarginine, polyornithine, protamine, spermine, spermidine, and putrescine. Many suitable methods for making such linkages are known in the art (see, for example, Ser. No.08/366,787, filed Dec. 30, 1994).

In an alternative embodiment, a metastatic marker subgenomic polynucleotide is associated with a liposome to form a gene delivery vehicle. Liposomes are small, lipid vesicles comprised of an aqueous compartment enclosed by a lipid bilayer, typically spherical or slightly elongated structures several hundred Angstroms in diameter. Under appropriate conditions, a liposome can fuse with the plasma membrane of a cell or with the membrane of an endocytic vesicle within a cell which has internalized the liposome, thereby releasing its contents into the cytoplasm. Prior to interaction with the surface of a cell, however, the liposome membrane acts as a relatively impermeable barrier which sequesters and protects its contents, for example, from degradative enzymes. Additionally, because a liposome is a synthetic structure, specially designed liposomes can be produced which incorporate desirable features. See Stryer, Biochemistry, pp. 236-240, 1975 (W. H. Freeman, San Francisco, Calif.); Szoka et al., Biochim. Biophys. Acta 600:1, 1980; Bayer et al., Biochim. Biophys. Acta. 550:464, 1979; Rivnay et al., Meth. Enzymol. 149:119, 1987; Wang et al., PNAS 84:7851, 1987, Plant et al., Anal. Biochem. 176:420, 1989, and U.S. Pat. No. 4,762,915. Liposomes can encapsulate a variety of nucleic acid molecules including DNA. RNA, plasmids, and expression constructs comprising metastatic marker subgenomic polynucleotides such those disclosed in the present invention.

Liposomal preparations for use in the present invention include cationic (positively charged), anionic (negatively charged) and neutral preparations. Cationic liposomes have been shown to mediate intracellular delivery of plasmid DNA (Felgner et al., Proc. Nat'l Acad. Sci. USA 84:7413-7416, 1987), mRNA (Malone et al., Proc. Nat'l Acad. Sci. USA 86:6077-6081, 1989), and purified transcription factors (Debs et al., J. Biol. Chem. 265:10189-10192, 1990), in functional form. Cationic liposomes are readily available. For example, N[1-2,3-dioleyloxy)propyl]-N,N,N-triethylammonium (DOTMA) liposomes are available under the trademark Lipofectin, from GIBCO BRL, Grand Island, N.Y. See also Felgner et al., Proc. Nat'l Acad Sci. USA 91: 5148-5152.87, 1994. Other commercially available liposomes include Transfectace (DDAB/DOPE) and DOTAP/DOPE (Boerhinger). Other cationic liposomes can be prepared from readily available materials using techniques well known in the art. See, e.g., Szoka et al., Proc. Nat'l Acad. Sci. USA 75:4194-4198, 1978; and WO 90/11092 for descriptions of the synthesis of DOTAP (1,2-bis(oleoyloxy)-3-(trimethylammonio)propane) liposomes.

Similarly, anionic and neutral liposomes are readily available, such as from Avanti Polar Lipids (Birmingham, Ala.), or can be easily prepared using readily available materials. Such materials include phosphatidyl choline, cholesterol, phosphatidyl ethanolamine, dioleoylphosphatidyl choline (DOPC), dioleoylphosphatidyl glycerol (DOPG), dioleoylphoshatidyl ethanolamine (DOPE), among others. These materials can also be mixed with the DOTMA and DOTAP starting materials in appropriate ratios. Methods for making liposomes using these materials are well known in the art.

The liposomes can comprise multilammelar vesicles (MLVs), small unilamellar vesicles (SUVs), or large unilamellar vesicles (LUVs). The various liposome-nucleic acid complexes are prepared using methods known in the art. See. e.g., Straubinger et al., METHODS OF IMMUNOLOGY (1983), Vol. 101, pp. 512-527; Szoka et al., Proc. Nat'l Acad Sci. USA 87:3410-3414, 1990; Papahadjopoulos et al., Biochim. Biophys. Acta 394:483, 1975; Wilson et al., Cell 17:77, 1979; Deamer and Bangham, Biochim. Biophys. Acta 443:629, 1976; Ostro et al., Biochem. Biophys. Res. Commun. 76:836, 1977; Fraley et al., Proc. Nat'l Acad. Sci. USA 76:3348, 1979; Enoch and Strittmatter, Proc. Nat'l Acad. Sci. USA 76:145, 1979; Fraley et al., J. Biol. Chem. 255:10431, 1980; Szoka and Papahadjopoulos, Proc. Nat'l Acad. Sci. USA 75:145, 1979; and Schaefer-Ridder et al., Science 215:166, 1982.

In addition, lipoproteins can be included with a metastatic marker subgenomic polynucleotide for delivery to a cell. Examples of such lipoproteins include chylomicrons, HDL, IDL, LDL, and VLDL. Mutants, fragments, or fusions of these proteins can also be used. Modifications of naturally occurring lipoproteins can also be used, such as acetylated LDL. These lipoproteins can target the delivery of polynucleotides to cells expressing lipoprotein receptors. Preferably, if lipoproteins are included with a polynucleotide, no other targeting ligand is included in the composition.

In another embodiment, naked metastatic marker subgenomic polynucleotide molecules are used as gene delivery vehicles, as described in WO 90/11092 and U.S. Pat. No. 5,580,859. Such gene delivery vehicles can be either metastatic marker DNA or RNA and, in certain embodiments, are linked to killed adenovirus. Curiel et al., Hum. Gene. Ther. 3:147-154, 1992. Other suitable vehicles include DNA-ligand (Wu et al., J. Biol. Chem. 264:16985-16987, 1989), lipid-DNA combinations (Felgner et al., Proc. Nat'l Acad. Sci. USA 84:7413 7417, 1989), liposomes (Wang et al., Proc. Nat'l Acad. Sci. 84:7851-7855, 1987) and microprojectiles (Williams et al., Proc. Nat'l Acad Sci. 88:2726-2730, 1991).

One can increase the efficiency of naked metastatic marker subgenomic polynucleotide uptake into cells by coating the polynucleotides onto biodegradable latex beads. This approach takes advantage of the observation that latex beads, when incubated with cells in culture, are efficiently transported and concentrated in the perinuclear region of the cells. The beads will then be transported into cells when injected into muscle. Metastatic marker subgenomic polynucleotide-coated latex beads will be efficiently transported into cells after endocytosis is initiated by the latex beads and thus increase gene transfer and expression efficiency. This method can be improved further by treating the beads to increase their hydrophobicity, thereby facilitating the disruption of the endosome and release of metastatic marker subgenomic polynucleotides into the cytoplasm.

The invention provides a method of detecting metastatic marker gene expression in a biological sample. Detection of metastatic marker gene expression is useful, for example, for identifying metastases or for determining metastatic potential in a tissue sample, preferably a tumor. Appropriate treatment regimens can then be designed for patients who are at risk for developing metastatic cancers in other organs of the body.

The body sample can be, for example, a solid tissue or a fluid sample. Protein or nucleic acid expression products can be detected in the body sample. In one embodiment, the body sample is assayed for the presence of a metastatic marker protein. A metastatic marker protein comprises a sequence encoded by a nucleotide sequence shown in SEQ ID NOS: 1-85 or its complement and can be detected using the marker protein-specific antibodies of the present invention. The antibodies can be labeled, for example, with a radioactive, fluorescent, biotinylated, or enzymatic tag and detected directly, or can be detected using indirect immunochemical methods, using a labeled secondary antibody. The presence of the metastatic marker proteins can be assayed, for example, in tissue sections by immunocytochemistry, or in lysates, using Western blotting, as is known in the art.

In another embodiment, the body sample is assayed for the presence of marker protein mRNA. A sample can be contacted with a nucleic acid hybridization probe capable of hybridizing with the mRNA corresponding the selected polypeptide. Still further, the sample can be subjected to a Northern blotting technique to detect mRNA, indicating expression of the polypeptide. For those techniques in which mRNA is detected, the sample can be subjected to a nucleic acid amplification process whereby the mRNA molecule or a selected part thereof is amplified using appropriate nucleotide primers. Other RNA detection techniques can also be used, including, but not limited to, in situ hybridization.

Marker protein-specific probes can be generated using the cDNA sequences disclosed in SEQ ID NOS: 1-85. The probes are preferably at least 15 to 50 nucleotides in length, although they can be at least 8, 10, 11, 12, 20, 25, 30, 35, 40, 45, 60, 75, or 100 or more nucleotides in length. The probes can be synthesized chemically or can be generated from longer polynucleotides using restriction enzymes. The probes can be labeled, for example, with a radioactive, biotinylated, or fluorescent tag.

Optionally, the level of a particular metastatic marker expression product in a body sample can be quantitated. Quantitation can be accomplished, for example, by comparing the level of expression product detected in the body sample with the amounts of product present in a standard curve. A comparison can be made visually or using a technique such as densitometry, with or without computerized assistance. For use as controls, body samples can be isolated from other humans, other non-cancerous organs of the patient being tested, or non-metastatic breast or colon cancer from the patient being tested.

Polynucleotides encoding metastatic marker-specific reagents of the invention, such as antibodies and nucleotide probes, can be supplied in a kit for detecting marker gene expression products in a biological sample. The kit can also contain buffers or labeling components, as well as instructions for using the reagents to detect the marker expression products in the biological sample.

If expression of a metastatic marker gene having a nucleotide sequence shown in SEQ ID NOS: 2, 4, 9, 13, 14, 19, 26, 29, 39-41, 48, 55, 57, 60, 63, 64, 72, 73, 82, or 83 is detected, the biological sample contains cancer cells which will likely metastasize to the lung. If expression of a metastatic marker gene having a nucleotide sequence shown in SEQ ID NOS: 1, 5, 11, 18, 20, 22, 24, 30, 33, 35, 36, 38, 45, 52, 58, 65, 66, 70, 74, 76, or 80 is detected, the biological sample contains cancer cells which will likely metastasize to the bone and/or lung. On the other hand, if expression of a metastatic marker gene having a nucleotide sequence shown in SEQ ID NOS: 3, 7, 8, 10, 12, 15-17, 21, 23, 25, 28, 31, 34, 37, 42-44, 46, 47, 49-51, 53, 59, 61, 62, 67,68, 75, 77-79, 81, 84, or 85 is detected, the biological sample contains cancer cells which will likely not metastasize. Detection of expression of a metastatic marker gene comprising the nucleotide sequence shown in SEQ ID NO: 56 also indicates that the biological sample contains cancer cells which will likely metastasize. This information can be used, for example, to design treatment regimens. Treatment regiments can include altering expression of one or more metastatic marker genes, as desired. Metastatic marker gene expression can be altered for therapeutic purposes, as described below, or can be used to identify therapeutic agents.

In one embodiment of the invention, expression of a metastatic marker gene whose expression is up-regulated in metastatic cancer is decreased using a ribozyme, an RNA molecule with catalytic activity. See, e.g., Cech, 1987, Science 236:1532-1539; Cech, 1990, Ann. Rev. Biochem. 59:543-568; Cech, 1992, Curr. Opin. Struct. Biol. 2: 605-609; Couture and Stinchcomb, 1996, Trends Genet. 12: 510-515. Ribozymes can be used to inhibit gene function by cleaving an RNA sequence, as is known in the art (e.g., Haseloff et al., U.S. Pat. No. 5,641,673).

Coding sequences of metastatic marker genes can be used to generate ribozymes which will specifically bind to mRNA transcribed from a metastatic marker gene. Methods of designing and constructing ribozymes which can cleave other RNA molecules in trans in a highly sequence specific manner have been developed and described in the art (see Haseloff, J. et al. (1988), Nature 334:585-591). For example, the cleavage activity of ribozymes can be targeted to specific RNAs by engineering a discrete “hybridization” region into the ribozyme. The hybridization region contains a sequence complementary to the target RNA and thus specifically hybridizes with the target (see, for example, Gerlach, W. L. et al., EP 321,201). Longer complementary sequences can be used to increase the affinity of the hybridization sequence for the target. The hybridizing and cleavage regions of the ribozyme can be integrally related; thus, upon hybridizing to the target RNA through the complementary regions, the catalytic region of the ribozyme can cleave the target.

Ribozymes can be introduced into cells as part of a DNA construct, as is known in the art. The DNA construct can also include transcriptional regulatory elements, such as a promoter element, an enhancer or UAS element, and a transcriptional terminator signal, for controlling the transcription of the ribozyme in the cells.

Mechanical methods, such as microinjection, liposome-mediated transfection, electroporation, or calcium phosphate precipitation, can be used to introduce a ribozyme-containing DNA construct into cells whose division it is desired to decrease, as described above. Alternatively, if it is desired that a DNA construct be stably retained by the cells, the DNA construct can be supplied on a plasmid and maintained as a separate element or integrated into the genome of the cells, as is known in the art.

As taught in Haseloff et al., U.S. Pat. No. 5,641,673, ribozymes can be engineered so that their expression will occur in response to factors which induce expression of metastatic marker genes. Ribozymes can also be engineered to provide an additional level of regulation, so that destruction of mRNA occurs only when both a ribozyme and a metastatic marker gene are expressed in the cells.

Expression of a metastatic marker gene can also be altered using an antisense oligonucleotide sequence. The antisense sequence is complementary to at least a portion of the coding sequence of a metastatic marker gene having a nucleotide sequence shown in SEQ ID NOS: 1-85. The complement of a nucleotide sequence shown in SEQ ID NOS: 1-85 is a contiguous sequence of nucleotides which form Watson-Crick basepairs with a contiguous nucleotide sequence shown in SEQ ID NOS: 1-85.

Preferably, the antisense oligonucleotide sequence is at least six nucleotides in length, but can be at least about 8, 12, 15, 20, 25, 30, 35, 40, 45, or 50 nucleotides long. Longer sequences can also be used. Antisense oligonucleotide molecules can be provided in a DNA construct and introduced into cells whose division is to be decreased, as described above.

Antisense oligonucleotides can comprise deoxyribonucleotides, ribonucleotides, or a combination of both. Oligonucleotides can be synthesized manually or by an automated synthesizer, by covalently linking the 5′ end of one nucleotide with the 3′ end of another nucleotide with non-phosphodiester internucleotide linkages such alkylphosphonates, phosphorothioates, phosphorodithioates, alkylphosphonothioates, alkylphosphonates, phosphoramidates, phosphate esters, carbamates, acetamidate, carboxymethyl esters, carbonates, and phosphate triesters. See Brown, 1994, Meth. Mol. Biol. 20:1-8; Sonveaux, 1994, Meth. Mol. Biol. 26:1-72; Uhlmann et al., 1990, Chem. Rev. 90:543-583.

Although precise complementarity is not required for successful duplex formation between an antisense molecule and the complementary coding sequence of a metastatic marker gene, antisense molecules with no more than one mismatch are preferred. One skilled in the art can easily use the calculated melting point of a metastatic marker gene antisense-sense pair to determine the degree of mismatching which will be tolerated between a particular antisense oligonucleotide and a particular coding sequence of the selected gene.

Antisense oligonucleotides can be modified without affecting their ability to hybridize to a metastatic marker protein coding sequence. These modifications can be internal or at one or both ends of the antisense molecule. For example, internucleoside phosphate linkages can be modified by adding cholesteryl or diamine moieties with varying numbers of carbon residues between the amino groups and terminal ribose. Modified bases and/or sugars, such as arabinose instead of ribose, or a 3′, 5′-substituted oligonucleotide in which the 3′ hydroxyl group or the 5′ phosphate group are substituted, can also be employed in a modified antisense oligonucleotide. These modified oligonucleotides can be prepared by methods well known in the art. Agrawal et al., 1992, Trends Biotechnol. 10:152-158; Uhlmann et al., 1990, Chem. Rev. 90:543-584; Uhlmann et al., 1987, Tetrahedron. Lett. 215:3539-3542.

Antibodies of the invention which specifically bind to a metastatic marker protein can also be used to alter metastatic marker gene expression. By antibodies is meant antibodies and parts or derivatives thereof, such as single chain antibodies, that retain specific binding for the protein. Specific antibodies bind to metastatic marker proteins and prevent the proteins from functioning in the cell. Polynucleotides encoding specific antibodies of the invention can be introduced into cells, as described above.

Marker proteins of the present invention can be used to screen for drugs which have a therapeutic anti-metastatic effect. The effect of a test compound on metastatic marker protein synthesis can also be used to identify test compounds which modulate metastasis. Test compounds which can be screened include any substances, whether natural products or synthetic, which can be administered to the subject. Libraries or mixtures of compounds can be tested. The compounds or substances can be those for which a pharmaceutical effect is previously known or unknown.

A cell is contacted with a test compound. The cell can be any cell, such as a colon cancer cell, which ordinarily synthesizes the metastatic marker protein being measured. For example, Tables 1 and 2 provide appropriate cell types which can be used for screening assays.

Synthesis of metastatic marker proteins can be measured by any means for measuring protein synthesis known in the art, such as incorporation of labeled amino acids into proteins and detection of labeled metastatic marker proteins in a polyacrylamide gel. The amount of metastatic marker proteins can be detected, for example, using metastatic marker protein-specific antibodies of the invention in Western blots. The amount of the metastatic marker proteins synthesized in the presence or absence of a test compound can be determined by any means known in the art, such as comparison of the amount of metastatic marker protein synthesized with the amount of the metastatic marker proteins present in a standard curve.

The effect of a test compound on metastatic marker protein synthesis can also be measured by Northern blot analysis, by measuring the amount of metastatic marker protein mRNA expression in response to the test compound using metastatic marker protein specific nucleotide probes of the invention, as is known in the art.

Typically, biological sample is contacted with a range of concentrations of the test compound, such as 1.0 nM, 5.0 nM, 10 nM, 50 nM, 100 nM, 500 nM, 1 mM, 10 mM, 50 mM, and 100 mM. Preferably, the test compound increases or decreases expression of a metastatic marker protein by 60%, 75%,. or 80%. More preferably, an increase or decrease of 85%, 90%, 95%, or 98% is achieved.

The invention provides compositions for increasing or decreasing expression of metastatic marker protein. Therapeutic compositions for increasing metastatic marker gene expression are desirable for markers which are down-regulated in metastatic cells. These compositions comprise polynucleotides encoding all or at least a portion of a metastatic marker protein gene expression product. Preferably, the therapeutic composition contains an expression construct comprising a promoter and a polynucleotide segment encoding at least a portion of the metastatic marker protein which is effective to increase or decrease metastatic potential. Portions of metastatic marker genes or proteins which are effective to decrease metastatic potential of a cell can be determined, for example, by introducing various portions of metastatic marker genes or polypeptides into metastatic cell lines, such as MDA-MB-231, MDA-MB-435, Km12C, or Km12L4, and assaying the division rate of the cells or the ability of the cells to form metastases when implanted in vivo, as is known in the art. Non-metastatic cell lines, such as MCF-7, can be used to assay the ability of a portion of a metastatic marker protein to increase expression of a metastatic marker gene.

Within the expression construct, the polynucleotide segment is located downstream from the promoter, and transcription of the polynucleotide segment initiates at the promoter. A more complete description of gene transfer vectors, especially retroviral vectors is contained in U.S. Ser. No. 08/869,309, which is incorporated herein by reference.

Decreased metastatic marker gene expression is desired in conditions in which the marker gene is up-regulated in metastatic cancer. Therapeutic compositions for treating these disorders comprise a polynucleotide encoding a reagent which specifically binds to a metastatic marker protein expression product, as disclosed herein.

Metastatic marker therapeutic compositions of the invention can comprise a pharmaceutically acceptable carrier. Pharmaceutically acceptable carriers are well known to those in the art. Such carriers include, but are not limited to, large, slowly metabolized macromolecules, such as proteins, polysaccharides, polylactic acids, polyglycolic acids, polymeric amino acids, amino acid copolymers, and inactive virus particles. Pharmaceutically acceptable salts can also be used in the composition, for example, mineral salts such as hydrochlorides, hydrobromides, phosphates, or sulfates, as well as the salts of organic acids such as acetates, proprionates, malonates, or benzoates.

Therapeutic compositions can also contain liquids, such as water, saline, glycerol, and ethanol, as well as substances such as wetting agents, emulsifying agents, or pH buffering agents. Liposomes, such as those described in U.S. Pat. No. 5,422,120, WO 95/13796, WO 91/14445, or EP 524,968 B1, can also be used as a carrier for the therapeutic composition.

Typically, a therapeutic metastatic marker composition is prepared as an injectable, either as a liquid solution or suspension; however, solid forms suitable for solution in, or suspension in, liquid vehicles prior to injection can also be prepared. A metastatic marker composition can also be formulated into an enteric coated tablet or gel capsule according to known methods in the art, such as those described in U.S. Pat. No. 4,853,230, EP 225,189, AU 9,224,296, and AU 9,230,801.

Administration of the metastatic marker therapeutic agents of the invention can include local or systemic administration, including injection, oral administration, particle gun, or catheterized administration, and topical administration. Various methods can be used to administer a therapeutic metastatic marker composition directly to a specific site in the body.

For treatment of tumors, including metastatic lesions, for example, a therapeutic metastatic marker composition can be injected several times in several different locations within the body of tumor. Alternatively, arteries which serve a tumor can be identified, and a therapeutic composition injected into such an artery, in order to deliver the composition directly into the tumor.

A tumor which has a necrotic center can be aspirated and the composition injected directly into the now empty center of the tumor. A therapeutic metastatic marker composition can be directly administered to the surface of a tumor, for example, by topical application of the composition. X-ray imaging can be used to assist in certain of the above delivery methods. Combination therapeutic agents, including a metastatic marker proteins or polypeptide or a metastatic marker subgenomic polynucleotide and other therapeutic agents, can be administered simultaneously or sequentially.

Receptor-mediated targeted delivery can be used to deliver therapeutic compositions containing metastatic marker subgenomic polynucleotides, proteins, or reagents such as antibodies, ribozymes, or antisense oligonucleotides to specific tissues. Receptor-mediated delivery techniques are described in, for example, Findeis et al. (1993), Trends in Biotechnol. 11, 202-05; Chiou et al. (1994), GENE THERAPEUTICS: METHODS AND APPLICATIONS OF DIRECT GENE TRANSFER (J. A. Wolff, ed.); Wu & Wu (1988), J. Biol. Chem. 263, 621-24; Wu et al. (1994), J. Biol. Chem. 269, 542-46; Zenke et al. (1990), Proc. Nat'l Acad. Sci. U.S.A. 87,3655-59; Wu et al. (1991), J. Biol. Chem. 266, 33842.

Alternatively, a metastatic marker therapeutic composition can be introduced into human cells ex vivo, and the cells then replaced into the human. Cells can be removed from a variety of locations including, for example, from a selected tumor or from an affected organ. In addition, a therapeutic composition can be inserted into non-affected, for example, dermal fibroblasts or peripheral blood leukocytes. If desired, particular fractions of cells such as a T cell subset or stem cells can also be specifically removed from the blood (see, for example, PCT WO 91/16116). The removed cells can then be contacted with a metastatic marker therapeutic composition utilizing any of the above-described techniques, followed by the return of the cells to the human, preferably to or within the vicinity of a tumor or other site to be treated. The methods described above can additionally comprise the steps of depleting fibroblasts or other non-contaminating tumor cells subsequent to removing tumor cells from a human, and/or the step of inactivating the cells, for example, by irradiation.

Both the dose of a metastatic marker composition and the means of administration can be determined based on the specific qualities of the therapeutic composition, the condition, age, and weight of the patient, the progression of the disease, and other relevant factors. Preferably, a therapeutic composition of the invention increases or decreases expression of the metastatic marker genes by 50%, 60%, 70%, or 80%. Most preferably, expression of the metastatic marker genes is increased or decreased by 90%, 95%, 99%, or 100%. The effectiveness of the mechanism chosen to alter expression of the metastatic marker genes can be assessed using methods well known in the art, such as hybridization of nucleotide probes to mRNA of the metastatic marker genes, quantitative RT-PCR, or detection of an the metastatic marker proteins using specific antibodies of the invention.

If the composition contains the metastatic marker proteins, polypeptide, or antibody, effective dosages of the composition are in the range of about 5 μg to about 50 μg/kg of patient body weight, about 50 μg to about 5 mg/kg, about 100 μg to about 500 μg/kg of patient body weight, and about 200 to about 250 μg/kg.

Therapeutic compositions containing metastatic marker subgenomic polynucleotides can be administered in a range of about 100 ng to about 200 mg of DNA for local administration. Concentration ranges of about 500 ng to about 50 mg, about 1 μg to about 2 mg, about 5 μg to about 500 μg, and about 20 μg to about 100 μg of DNA can also be used during a gene therapy protocol. Factors such as method of action and efficacy of transformation and expression are considerations that will affect the dosage required for ultimate efficacy of the metastatic marker subgenomic polynucleotides. Where greater expression is desired over a larger area of tissue, larger amounts of metastatic marker subgenomic polynucleotides or the same amounts readministered in a successive protocol of administrations, or several administrations to different adjacent or close tissue portions of, for example, a tumor site, can be required to effect a positive therapeutic outcome. In all cases, routine experimentation in clinical trials will determine specific ranges for optimal therapeutic effect.

Expression of an endogenous metastatic marker gene in a cell can also be altered by introducing in frame with the endogenous metastatic marker gene a DNA construct comprising a metastatic marker protein targeting sequence, a regulatory sequence, an exon, and an unpaired splice donor site by homologous recombination, such that a homologously recombinant cell comprising the DNA construct is formed. The new transcription unit can be used to turn the metastatic marker gene on or off as desired. This method of affecting endogenous gene expression is taught in U.S. Pat. No. 5,641.670, which is incorporated herein by reference.

The targeting sequence is a segment of at least 10, 12, 15, 20, or 50 contiguous nucleotides selected from the nucleotide sequences shown in SEQ ID NOS: 1-85 or the complements thereof. The transcription unit is located upstream of a coding sequence of the endogenous metastatic marker protein gene. The exogenous regulatory sequence directs transcription of the coding sequence of the metastatic marker genes.

A metastatic marker subgenomic polynucleotide can also be delivered to subjects for the purpose of screening test compounds for those which are useful for enhancing transfer of metastatic marker subgenomic polynucleotides to the cell or for enhancing subsequent biological effects of metastatic marker subgenomic polynucleotides within the cell. Such biological effects include hybridization to complementary metastatic marker mRNA and inhibition of its translation, expression of a metastatic marker subgenomic polynucleotide to form metastatic marker mRNA and/or metastatic marker protein, and replication and integration of a metastatic marker subgenomic polynucleotide. The subject can be a cell culture or an animal, preferably a mammal, more preferably a human.

Test compounds which can be screened include any substances, whether natural products or synthetic, which can be administered to the subject. Libraries or mixtures of compounds can be tested. The compounds or substances can be those for which a pharmaceutical effect is previously known or unknown. The compounds or substances can be delivered before, after, or concomitantly with a metastatic marker subgenomic polynucleotide. They can be administered separately or in admixture with a metastatic marker subgenomic polynucleotide.

Integration of a delivered metastatic marker subgenomic polynucleotide can be monitored by any means known in the art. For example, Southern blotting of the delivered metastatic marker subgenomic polynucleotide can be performed. A change in the size of the fragments of a delivered polynucleotide indicates integration. Replication of a delivered polynucleotide can be monitored inter alia by detecting incorporation of labeled nucleotides combined with hybridization to a metastatic marker probe. Expression of metastatic marker subgenomic polynucleotide can be monitored by detecting production of metastatic marker mRNA which hybridizes to the delivered polynucleotide or by detecting metastatic marker protein. Metastatic marker protein can be detected immunologically. Thus, the delivery of metastatic marker subgenomic polynucleotides according to the present invention provides an excellent system for screening test compounds for their ability to enhance transfer of metastatic marker subgenomic polynucleotides to a cell, by enhancing delivery, integration, hybridization, expression, replication or integration in a cell in vitro or in an animal, preferably a mammal, more preferably a human.

The above disclosure generally describes the present invention. A more complete understanding can be obtained by reference to the following specific examples which are provided herein for purposes of illustration only, and are not intended to limit the scope of the invention.

### EXAMPLE 1

### Differentially Expressed Genes

This example demonstrates polynucleotides that are differentially expressed in human breast or colon cancer cell lines.

Human cell lines used to identify differentially expressed polynucleotides are the human breast cancer cell lines MCF-7 (non-metastatic), MDA-MB-231 (metastatic to bone and/or lung), and MDA-MB-435 (metastatic to lung) (Brinkley and Cailleau, 1980, Cancer Res. 40:3118), and the colon cancer cell lines Km12C (low metastatic) and Km12L4A (highly metastatic) (Morikawa et al., 1988, Cancer Res. 48:1943-1948).

RNA was prepared from each cell line and reverse transcribed to form cDNA. The cDNA was amplified using random primers. Amplification products were visualized on a sequencing gel, and cDNA corresponding to mRNA which was differentially expressed in the cell lines was identified.

Expression patterns and sequence identification numbers of novel metastatic marker polynucleotides are shown in Table 1.

Expression patterns and sequence identification numbers of metastatic marker polynucleotides which correspond to known genes are shown in Table 2, and the corresponding proteins are described below.

Osteopontin (SEQ ID NO: 64) (OPN or Spp1 for secreted phosphoprotein 1) is a secreted extracellular matrix protein, often expressed during wound healing, involved in osteoclastic differentiation and activation, as described in Heymann et al., 1998, Cytokine 10:155-168. Osteopontin is found in bone and other epithelial cells, and has been shown to stimulate proliferation of a quiescent subpopulation of prostate epithelial cells (see Elgavish et al., 1998, Prostate 35:83-94).

Osteopontin is implicated during the development of diabetic nephropathy (Fischer et al., 1998, Diabetes 47:1512-1518); the process of cartilage-to-bone transition during rigid bone healing after bone fracture (Nakase et al., 1998, Acta Histochem 100:287-295); wound healing by an interaction with the receptor integrin alpha(v)beta 3 after focal stroke (Ellison et al., 1998, Stroke 29:1698-1706); integrin receptor binding and signaling during cell attachment and mechanical stimulation of osteoblasts (Carvalho et al., 1998, J. Cell Biochem 70:376-390); kidney morphogenesis (Denda et al., 1998, Mol. Biol. Cell 9:1425-1435); and as an interstitial chemoattractant in renal inflammation (Rovin and Phan, 1998, Am. J. Kidney Dis. 31:1065-1084). Mice lacking the osteopontin gene showed modulation in osteoclast differentiation from wild type mice (see Rittling et al., 1998, J. Bone Miner Res. 13: 1101-1111).

Osteopontin is synthesized by monocytes and macrophages within injury sites, and can promote leukocyte adhesion through the alpha 4beta1 integrin, as described in Bayless et al., 1998, J. Cell Sci. 111:1165-1174. Osteopontin is transcriptionally regulated by retinoic acid (see Manji et al., 1998, J. Cell Physiol. 176:1-9); preferentially expressed in high grade metastatic brain tumors compared to low grade brain tumors, and inducible by tissue plasminogen activator (tPA) in glioma cell lines (see Tucker et al., 1998, Anticancer Res. 18:807-812). Osteopontin is expressed in about 73% of primary gastric carcinoma tissues and correlated with the progression of human gastric carcinoma and lymphogenous metastasis (see Ue et al., 1998, Int. J. Cancer 79:127-132).

Nip (SEQ ID NO: 65) is described in Boyd et al., 1994, Cell 79:341-351. Adenovirus E1B 19 kDa protein protects against cell death induced by viral infection and external stimuli, and can be functionally substituted with the Bcl-2 protoncogene. E1B 19 kDa interacting proteins (Nip1, Nip2, and Nip3) were discovered in yeast two-hybrid studies conducted to discern proteins that interact with 19 kDa protein, as described by Boyd et al., supra. Nip 1, 2, and 3 interact with discrete domains of E1B 19 kDa, and similarly also interact with Bcl-2, in both cases promoting cell survival.

Ca-dependent protease (SEQ ID NO: 66) is Ca⁺²-dependent protease (also called calpain), activity of which is present in every vertebrate cell that has been examined. Ca⁺²-dependent protease activity is associated with cleavages that alter regulation of various enzyme activities, with remodeling or disassembly of the cell cytoskeleton, and with cleavages of hormone receptors (see Goll et al., 1992, Bioessays 14(8):549-556). Ca⁺²-dependent protease activity is regulated by binding of Ca⁺² to specific sites on the calpain molecule, with binding to each site generating a specific response corelated with a specific activity (e.g., proteolytic activity, calpastatin binding, etc.), as described in Goll et al. Excessive activation of the Ca⁺²-dependent protease calpain may play a role in the pathology of disorders including cerebral ischemia, cataract, myocardial ischemia, muscular dystrophy, and platelet aggregation. Therapeutic applications include selective Ca⁺²-dependent protease inhibition, as described in Wang and Yuen, 1994, Trends Pharmacol. Sci. 15(11):412-419.

IGF-R (insulin-like growth factor receptor) (SEQ ID NO: 67) is a transmembrane tyrosine kinase linked to the ras-raf-MAPK(mitogen-activated protein kinase) cascade and required for the cell to progress through the cell cycle (Werner and Roith, 1997, Crit. Rev. Oncog 8(1):71-92) IGF-R mediates mitogenesis, growth hormone action, cell survival and transformation to and maintenance of the malignant phenotype. IGF-R is a member of the growth factor receptor tyrosine kinase superfamily, exists as covalent cross-linked dimers where each monomer is composed of two subunits, and is bound by ligand in the extracellular domain (McInnes and Sykes, 1997, Biopolymers 43(5):339-366).

The domains of the IGF-R are described in Sepp-Lotenzino, 1998, Breast Cancer Res Treat 47(3):235-253, including domains responsible for mitogenesis, transformation, and protection from apoptosis. IGF-R expression is increased in breast cancer cells derived from tumor tissue and cell lines, as described in Surmacz et al., 1998, Breast Cancer Res Treat 47(3):255-267, and increased IGF-R may increase tumor mass and/or aid tumor recurrence by promoting proliferation, cell survival, and cell-cell interactions. Human pancreatic cancers overexpress IGF-R and its ligand (Korc, 1998, Surg Oncol Clin N Am 7(1): 2541), and expression of IGF-I and IGF-R is determined to be a prognostic factor (reflecting the interaction between the neoplastic cells and their microenvironment) for lymphocytic infiltration in thryoid carcinomas (Fonseca et al., 1997, Verh Dtsch Ges Pathol 81:82-96).

ILGF-BP5 (SEQ ID NO: 68) is insulin-like growth factor binding protein 5, described in Allander et al., 1994, J. Biol. Chem. 269:10891-10898. The gene and promoter for IGF-BP5 are characterized by Allander et al., 1994, J. Biol Chem. 269:10891-10898, and some general actions of IGF-BPs are described in Chan and Spencer, 1997, Endocrine 7:95-97. Potential impact of IGF-BPs on cancer cell growth is described in Oh, 1997, Endocrine 7:111-113, and Oh, 1998, Breast Cancer Res Treat 47:283-293. IGF-BP5 is expressed during brain development: IGF-BP5 and IGF-1 mRNAs are synchronously coexpressed in principal neurons of sensory relay systems, including the olfactory bulb, medial and dorsal lateral geniculate bodies, and ventral tier, cochlear, lemniscal, and vestibular nuclei, and are transiently coexpressed in principal neurons of the anterodorsal nucleus, as described in Bondy and Lee, 1993, J. Neurosci 13(12):5092-5104. IGF-BP5 is expressed by luminal or cumulus granulosa cells in virtually all follicles, and is highly abundant in stromal interstitial cells of the mature ovary (see Zhou and Bondy, 1993, Biol. Reprod 48:467-482). IGF-BP5 induction is strongly stimulated during differentiation of skeletal myoblasts and is correlated with IGF-R activation as described in Rousse et al., 1998, Endocrinology 139:1487-1493. IGF-BP5 and other components of the IGF system are critical in postnatal brain development (see Lee et al., 1996, J. Cereb Blood Flow Metab 16:227-236).

IGF-BP5 stimulates bone cell proliferation by an IGF-independent mechanism involving IGF-BP5-specific cell surface binding sites, as described in Mohan et al., 1995, J. Biol Chem 270:20424-20431. In connective tissue cell types, IGF-BP5 has a lowered binding affinity to the extracellular matrix which allows IGF-I to better equilibrate with the receptors which in turn potentiates IGF-I action on fibroblasts and smooth muscle cells (Clemmons, Mol Cell Endocrinology 140:19-24).

Lactate dehydrogenase (SEQ ID NO: 69) is a member of the LDH group of tetrameric enzymes with five isoforms composed of combinations of two subunits, LDH-A and LDH-B. Shim et al., 1997, Proc. Nat'l Acad. Sci. 94:6658-6663, described the relationship between LDH-A and neoplasia. In particular, overexpression on LDH-A may contribute to altered metabolism that confers neoplastic growth advantage. The expression pattern of LDH in the present invention is consistent, in that LDH expression is higher in two metastatic breast cancer cell lines than in a non-metastatic breast cancer cell line (Table 2). High or increasing lactate dehydrogenase (LDH) levels in tumor tissue and cells is associated with poor survival rate in small cell lung carcinoma (SCLC), as described in Ray et al., 1998. Cancer Detect Prev 22:293-304, making it a useful prognostic indicator for SCLC as discussed in Stokkel et al., 1998, J. Cancer Res Clin Oncol 124:215-219.

Ufo TKR (SEQ ID NO: 70) is described in Schulz et al., 1993, Oncogene 8:509-513. This protein has been reported as a marker in tumors, but has not previously been reported in breast cancer. According to the present invention, expression is found in the MDA-MB-231 breast cancer cell line, but not in the MSF-7 or MDA-MB-435 cell lines. This gene and protein provide new markers for distinguishing breast cancer tissue of different types of metastatic potential.

Initially isolated from primary human myeloid leukemia cells, the ufo oncogene (also called Axl or Ark) is a receptor tyrosine kinase (RTK). Its genomic structure is described in Schulz et al., supra, and its differential expression is described in Challier et al., 1996. Leukemia 10:781-787. The ufo protein is a member of a class of RTKs having two fibronectin type III domains and two immunoglobulin-like domains present in the extracellular portion, and is preferentially expressed in monocytes, stromal cells, and some CD34-positive progenitor cells (Neubauer et al., 1997, Leuk Lymphoma 25:91-96). Ufo has an extracellular structure similar to neural cell adhesion molecules, and has direct or indirect binding sites for PLCgamma, GRB2, c-src, and lck (Braunger et al., 1997, Oncogene 14:2619-2631).

eIF-2 (SEQ ID NO: 71) is a translation initiation factor, and functions as a heterotrimeric GTP-binding protein involved in the recruitment of methionyl-tRNA to the 40 S ribosomal subunit (Gasper et al., 1994, J. Biol. Chem. 269:3415-3422). According to the present invention, higher expression is found in two metastatic breast cancer cell lines and not in cell line MCF-7.

eIF-2 is involved in introducing the initiator tRNA into the translation mechanism and performing the first step in the peptide chain elongation cycle. eIF-2 is associated with a 5 subunit molecule having GTP recycling function called eIF-2B (Kyrpides and Woese, 1998, Proc. Nat'l Acad. Sci. USA 95:3726-3730, and Kimball et al., 1998, J. Biol. Chem. 273:12841-12845).

eIF-2 has subunits alpha and beta. eIF-2alpha is phosphorylated at Ser 51 and then modulates the interaction of eIF-2 and eIF-2B, as described in Kimball et al., 1998, Protein Expr. Purif. 12:415-419, Kimball et al., 1998, J. Biol. Chem. 273:3039-3044, and Pavitt 1998, Genes Dev. 12:514-526. It is reported that by regulating translation initiation, control of cell growth and division in eukaryotic cells is achieved: for example, clotrimazole, a potent anti-proliferative agent in vitro and in vivo, depletes intracellular Ca⁺² stores, which activates PKR, resulting in the phosphorylation of eIF-2alpha, and the ultimate inhibition of protein synthesis and blockage of the cell cycle in G1 phase (Aktas et al., 1998, Proc. Nat'l Acad. Sci. USA 95:8280-8285). Additionally, Kim et al., 1998, Mol. Med. 4:179-190, show that nitric oxide (NO) suppresses protein synthesis in cell types including human ovarian tumor cells by stimulating phosphorylation of eIF-2alpha.

Glutaminyl cyclase (SEQ ID NO: 72) is described by Song et al., 1994, J. Mol. Endocrinol. 13:77-86, and is expressed most highly in the most metastatic cell line MDA-MB-435, as compared to less metastatic line MDA-MB-231 and non-metastatic line MCF-7. Glutaminyl cyclase (also called glutamine cyclotransferase) converts glutaminyl-peptides (such as gonadotropin-releasing hormone and thyrotropin-releasing hormone) into pyroglutamyl-peptides, as described in Busby et al., 1987, J. Biol. Chem. 262:8532-8536, Fischer and Spiess, 1987. Proc. Nat'l Acad. Sci. USA 84:3628-3632, and Pohl et al., 1991, Proc. Nat'l Acad. Sci. 88:10059-10063. Cloning and sequence analysis of glutaminyl cyclase derived from a human pituitary cDNA library is described in Song et al., 1994. J. Mol. Endocrinol. 13:77-86. Studies on the catalytic pathway of glutaminyl cyclase and its substrate specificity are described in Gololobov et al., 1996, Biol. Chem. Hoppe Seyler 377:395-398. Assays for the presence of glutaminyl cyclase activity are described in Koger et al., 1989, Method Enzymol. 168:358-365 and Houseknecht et al., 1998, Biotechniques 24:346.

gp130 (SEQ ID NO: 73) is transmembrane protein glycoprotein 130. gp130 is a signal transducing shared component of the receptor complexes for the interleukin-6 (IL-6)-type cytokines (Hirano et al., 1997, Cytokine Growth Factor Rev. 8:241-252), including IL-6, IL-11, leukemia inhibitor factor (LIF), oncostatin M (OSM), ciliary neurotrophic factor and cardiotrophin-1. The N-terminal of gp130 is an extracellular immunoglobulin-like portion of the protein (Hammacher et al., 1998, J. Biol. Chem. 273:22701-22707). Signal transduction including gp130 occurs through the gp130/Jak/STAT pathway 1 (Heinrich 1998, Biochem. J. 334:297-314). The cytokines acting through the pathway that includes gp130 (also called gp130 cytokines) exhibit pleitropic biological activities including immune, hematopoietic, and neural effects (Nakashima and Taga, 1998, Semin Hematol. 35:210-221, Thompson et al., 1998, Neuroscience 84:1247-1255, Hirano, 1998, Int. Rev. Immunol. 16:249-284, Marz et al., 1997, Eur. J. Neurosci. 9:2765-2773, and Betz and Muller, 1998, Int Immunol 10:1175-1184).

gp130 cytokines are reported to control survival and proliferation of myeloma cell lines and primary myeloma cells (Klein, 1998, Curr. Opin. Hematol. 5:186-191). gp130 is expressed in the majority of renal cell carcinomas and has an important role in the proliferation of some renal cell carcinoma cell lines (Costes et al., 1997, J. Clin. Pathol. 50:835-840).

E-cadherin (SEQ ID NO: 75) is a member of a family of glycoproteins responsible for calcium-dependent cell-cell adhesion and is implicated in maintaining cytoskeletal integrity. Epithelial cadherin (E-cadherin) mediated cell adhesion system in cancer cells is inactivated by multiple mechanisms corresponding to the pathological features of the particular tumor type (Hirohashi, 1998, Am J Pathol 153:333-339). In general the cadherin system mediates Ca⁺²-dependent homophilic cell-cell adhesion. Transcriptional inactivation of E-cadherin expression occurs frequently in tumor progression, and thus inactivation or downregulation of E-cadherin plays a significant role in multistage carcinogenesis (Hirohashi, 1998, Am J Pathol 153:333-339).

E-cadherin is characterized as a tumor suppressor of the metastatic phenotype, as described in MacGrogan and Bookstein, 1997, Semin Cancer Biol 8:11-19, and cadherins are important determinants of tissue morphology including invasive carcinoma as described in van der Linden, 1996, Early Pregnancy 2:5-14, and Yap, 1998, Cancer Invest. 16:252-261.

Mechanisms of action of cadherins are discussed in Daniel and Reynolds, 1997, Bioessays 19:883-891. The structure and function of cell adhesion molecules including E-cadherin are described in Joseph-Silverstein and Silverstein, 1998, Cancer Invest. 16:176-182, Yap et al., 1997, Annu. Rev. Cell Dev. Biol. 13:119-146, and Uemura, 1998, Cell 93:1095-1098. Cell adhesion molecules including E-cadherin are potential targets for anti-cancer drugs and therapeutics to treat acute or chronic inflammatory disease as described in Buckley and Simmons, 1997, Mol Med Today 3:449-456, Moll and Moll, 1998, Virchows Arch 432:487-504.

According to the present invention, E-cadherin is expressed in non-metastatic breast cancer cell line MCF-7, and not in MDA-MB-231 and MDA-MB435. The expression products are diagnostic markers indicating the metastatic potential of breast cancer tissue samples.

Serpin (SEQ ID NO: 76), serine protease inhibitors, are a family of protease inhibitors that inhibit chymotrypsin-like serine proteases (Whisstock et al., 1998, Trends Biochem. Sci. 23:63-67) and that have the unique ability to regulate their activity by changing the conformation of their reactive-center loop; studies of serpin variants provide definition for the functional domains of serpins that control the folding and link serpins mutations to disease (see Stein and Carrell, 1995, Nat. Struct. Biol. 2:96-113). Serine protease cleavage of proteins is essential to a wide variety of biological processes, and the cleavage is primarily regulated by the cleavage inhibitors, as described in Wright, 1996, Bioessays 18:453-464. Members of the serpin family include alpha 1-antitrypsin (AAT) (Carrell et al., 1996, Chest 110:243S-247S), alpha2-anti-plasmin (PAI-1 and PAI-2) (Andreasen et al., 1997, Int. J. Cancer 72:1-22), thrombin, urokinase plasminogen activator, and kallikrein (Turgeon and Houenou, 1997, Brain Res Brain Res Rev 25:85-95). Some serpins also have other activities including neuronal differentiating and survival activities (Becerra, 1997, Adv. Exp. Med. Biol. 425:332-237) and tumor suppression (Sager et al., 1997, Adv. Exp. Med. Biol. 425:77-88). PAI-1 and PAI-2 are linked to cancer metastasis, as described in Andreasen et al., 1997, Int. J. Cancer 72:1-22.

pS2 (SEQ ID NO: 77) was isolated from MCF7 human breast cancer cells, as described in Takahashi et al., 1990, FEBS Letters 261:283-286. pS2 is estrogen-regulated. Speiser et al., 1997, Anticancer Research 17:679-684, reported that the pS2 status declined from well to poorly differentiated ovarian cancer. pS2 expression also is associated with a good prognosis in breast cancer patients. According to the present invention, pS2 is expressed in MCF-7 cells, but not in two metastatic breast cancer cell lines

pS2 (presenilin-2 or trefoil factor 1 (TFF 1)) is a trefoil polypeptide normally expressed in the mucosa of the gastrointestinal tract, and found ectopically in gastrointestinal inflammatory disorders and various carcinomas (May and Westley, 1997, J. Pathol. 183:4-7. pS2 is expressed in breast cancers (Poulsom et al., 1997, J. Pathol. 183:30-38). pS2 is a pleitropic factor involved in mucin polymerization, cell motility (Modlin and Poulsom, 1997, J. Clin. Gastroenterol 25(1):S94-S100), cell proliferation and/or differentiation, and possibly in the nervous system (see Ribieras et al., 1998, Biochim. Biophys. Acta. 1378:F61-F77).

LIV-1 (SEQ ID NO: 78) is an estrogen-regulated protein reported in the MCF-7 cell line (Green et al., GeneBank submission Accession No. U41060). According to the present invention, LIV-1 is expressed in MCF-7 cells, but not in two metastatic breast cancer cell lines.

Leucine-isoleucine-valine-1 (LIV-1) and other members of the LIV family (LIV-2, 3, and 4) are binding proteins that represent a transport system for branched chain amino acids in E. coli as described in Yamamoto et al., 1979, J. Bacteriol. 138:24-32, and Yamamoto and Anraku, 1980, J. Bacteriol. 144:36-44. A human homologue to LIV-1 is both estrogen and growth factor inducible in MCF-7 human breast cancer cell line (El-Tanani and Green, 1997, J. Steroid. Biochem. Mol. Biol 60:269-276; El-Tanani and Green, 1996, Mol Cell Endocrinol 124:71-77; and El-Tanani and Green, 1996, Mol Cell Endocrinol 121:29-35).

GTP-binding protein (SEQ ID NO: 79) is a member of the family of guanine nucleotide-binding regulatory proteins, G proteins. The protein is expressed in MCF-7 cells, but not in two metastatic breast cancer cell lines.

G proteins provide signaling mechanisms for the serpentine family of receptors as described in Dhanasekaran and Prasad, 1998, Biol. Signals Recept 7:109-117. Studies indicate that the alpha as well as the beta gamma subunits of the GTP-binding proteins are involved in the regulation of several cellular responses, some of which responses are critical to the regulation of cell growth and differentiation (Dhanasekaran and Prasad, 1998, Biol Signals Recept 7:109-117). G protein coupled receptors regulate the mitogen activated protein kinase pathway as described in Russell and Hoeffler, 1996, J. Invest. Dermatol Symp Proc 1:119-122, and thus play a role in controlling cell growth. GTP binding proteins are also implicated in the regulation of intracellular transport as described in Ktistakis, 1998, Bioessays 20:495-504.

Chemokines induce various intracellular signaling pathways in natural killer cells by activating members of GTP binding proteins as described in Maghazachi and Al-Auokaty, 1998, FASEB J. 12:913-924. Heterotrimeric GTP binding proteins regulate distinct signaling pathways, some of which in turn regulate the activity of Na+/H+exchanger proteins as described in Voyno-Yasenetskaya, 1998, Biol Signals Recept 7:118-124.

Desmoplakin (SEQ ID NO: 84) is a member of a family of proteins that serve as cell surface attachment sites for cytophasmic intermediate filaments.

Vimentin (SEQ ID NO: 80) is a member of the intermediate filament gene family (Evans, 1998, Bioessays 20:79-86. Intermediate filaments are a major component of the cytoskeleton of higher eukaryotes. Vimentin gene knockout mice indicate degeneration of the cerebellar Purkinje cells (Galou et al., 1997, Biol Cell 89:85-97). Vimentin is positive in immunohistochemical reactions of sarcomas and related lesions (Gaudin et al., 1998, Am J Surg Pathol 22:148-162), and of desmoplastic small round-cell tumors and their variants (Gerald et al., 1998, J. Clin. Oncol. 16:3028-3036). Vimentin is also expressed in neoplasms showing follicular dendritic cell differentiation as described in Perez-Ordonez and Rosai, 1998, Semin. Diagn. Pathol. 15:144-154, and in biphasic carcinomatous-sarcomatous malignant mixed mullerian tumors as described in Guarino et al., 1998. Tumori 84:391-397.

Cytochrome C Oxidase (CcO) (SEQ ID NO: 81) is the terminal enzyme of the respiratory chain of mitochondria and aerobic bacteria: it catalyzes electron transfer from cytochrome C to molecular oxygen, reducing the oxygen to water (Michel et al., 1998, Annu Rev Biophys Biomol Struct 27:329-356). Cytochrome C oxidase is a member of the superfamily of quinol and cytochrome C oxidase complexes that are related by a homologous subunit containing six positionally conserved histidines that ligate a low-spin heme and a heme-copper dioxygen activating and reduction center as described in Musser and Chan, 1998, J. Mol. Evol. 46.508-520. Cytochrome C and ubiquinol oxidases are membrane-bound redox-driven proton pumps which couple an electron current to a proton current across the membrane (see Karpefors et al., 1998, Biochim Biophys Acta 1365:159-169). Analysis of mutant forms of cytochrome C oxidase is described in Mills and Ferguson-Miller, 1998, Biochim Biophys Acta 365:46-52. Nitric oxide inhibits respiration at cytochrome C oxidase, as described in Torres et al., 1998, J. Bioenerg Biomembr 30:63-69.

Heat shock protein 90 (hsp90) (SEQ ID NO: 82) acts as a chaperone molecule in association with the glucocorticoid and progesterone nuclear receptors, and has A, B, and Z regions for facilitating these interactions (Dao-Phan et al., 1997, Mol Endocrinol 11:962-972). Levels of hsp90 are reported elevated in active systemic lupus erythematosus (Stephanou et al., 1997, Biochem J 321:103-106). Increased hsp90 expression is implicated in regulation of forms of cell injury that lead to programmed cell death as described in Galea-Lauri et al., 1996, J. Immunol. 157:4109-4118. Hsp90 is upregulated in regenerating fibers and diseased fibers of Duchenne muscular dystrophy (Bornman et al., 1996, Muscle Nerve 19:574-580), and is a candidate substrate for proteolysis during ionizing radiation-induced apoptosis of some breast cancer cells (Prasad et al., 1998, Int. J. Oncol 13:757-764). Hsp90 is involved in dislocation of the mutant insulin receptors from the endoplasmic reticulum to the cytosol as described in Imamura et al., 1998, J. Biol. Chem. 273:11183-11188, and associates with and activates endothelial nitric oxide synthase as described in Garcia-Cardena et al., 1998, Nature 392:821-824.

Integrin alpha 6 (SEQ ID NO: 83) is in the family of integrins, heterodimeric, cation dependent cell membrane adhesion molecules that mediate cell-cell and cell-matrix interactions. Integrin alpha 6 is a component of the hemidesmosome complex (Jones et al., 1998, Bioessays 20:488-494). Integrins maintain tissue integrity and regulate cell proliferation, growth, differentiation, and migration. (See Thomas et al., 1997, Oral Oncol 33:381-388). In oral squamous cell carcinomas there is a variable loss or reduced expression of integrin alpha 6, as described in Thomas et al., 1997, Oral Oncol. 33:381-388. Alpha 6 integrin also plays an active role in invasion of intestinal and diffuse-type cells of representative gastric carcinoma cell lines as described in Koike et al., 1997, J. Cancer. Res. Clin. Oncol. 123L:310-316.

Osteogenic protein-1 (OP-1) (also called BMP-7) (SEQ ID NO: 85) is a morphogenetic factor (and a member of the bone morphogenetic protein (BMP) family of growth factors) and is highly expressed in kidney and involved in tissue repair and development (see Almanzar et al., 1998, J. Am. Soc. Nephrol. 9:1456-1463). OP-1 is also expressed in the developing nervous system and can induce dendritic growth in sympathetic neurons as described in Guo et al., 1998, Neurosci. Lett 245:131-134. OP-1 stimulates cartilage formation as described in Klein-Nulend et al., 1998, J. Biomed. Mater. Res. 40:614-620.

OP-1 induces down-regulation of insulin-like growth factor binding proteins (particularly IGFBP-5) thus affecting IGF-1 in the context of bone cell differentiation and mineralized bone nodule formation as described in Yeh et al., 1997, Endocrinology 138:4181-4190. OP-1 can be used as a bone graft substitute to promote spinal fusion and to aid in the incorporation of metal implants (Cook and Rueger, 1996, Clin. Orthop. 324:29-38). The three dimensional structure of OP-1 is reported in Griffith et al., 1996, Proc Nat'l Acad Sci 93:878-883.

The protein encoded by SEQ ID NO: 56 is a putative secreted protein and is highly expressed in fat tissue.

TABLE 1
Novel Differentially Expressed Metastatic Marker Polynucleotides

| | | | breast | | | |
|---|---|---|---|---|---|---|
| | | | cancer | breast | | |
| | | | metastatic | cancer | low | |
| | | non- | to bone | metastatic | metastatic | high |
| | | metastatic | and/or lung | to lung | from | metastatic |
| TRANSCRIPT | SEQ ID | breast | MDA-MB- | MDA- | colon | from colon |
| NUMBER | NO: | MCF-7 | 231 | MB-435 | KM12C | KM12L4A |
| 901 | 1 | − | + | − | | |
| 907 | 2 | − | − | + | | |
| 9102b | 3 | + | − | − | | |
| 9114 | 4 | − | − | + | | |
| 9121a | 5 | − | + | − | | |
| 9129 | 6 | + | − | + | | |
| 9139a | 7 | + | − | − | | |
| 9143b | 8 | + | − | − | | |
| 9157b | 9 | − | − | + | | |
| 9166 | 10 | + | − | − | | |
| 9170b | 11 | − | + | − | | |
| 9190a | 12 | + | − | − | | |
| 9191 | 13 | − | − | + | | |
| 9216 | 14 | − | − | + | | |
| 9224c | 15 | + | − | − | | |
| 9230b | 16 | + | − | − | | |
| 924 | 17 | + | − | − | | |
| 9242a | 18 | − | + | − | | |
| 9259a | 19 | − | − | + | | |
| 9261 | 20 | − | + | − | | |
| 9272 | 21 | + | − | − | | |
| 9293b | 22 | − | + | − | | |
| 9304b | 23 | + | − | − | | |
| 9307a | 24 | − | + | − | | |
| 931 | 25 | + | − | − | | |
| 9313 | 26 | − | − | + | | |
| 9316 | 27 | + | + | − | | |
| 9318b | 28 | + | − | − | | |
| 9320a | 29 | − | − | + | | |
| 9330b | 30 | − | + | − | | |
| 9335 | 31 | + | − | − | | |
| 9337 | 32 | + | − | + | | |
| 9342b | 33 | − | + | − | | |
| 9343c | 34 | + | − | − | | |
| 9350e | 35 | − | + | − | | |
| 9351b | 36 | − | + | − | | |
| 9361 | 37 | + | − | − | | |
| 9368 | 38 | − | + | − | | |
| 9373b | 39 | − | − | + | | |
| 9385a | 40 | − | − | + | | |
| 9386c | 41 | − | − | + | | |
| 9388d | 42 | + | − | − | | |
| 9390 | 43 | + | − | − | | |
| 9393 | 44 | + | − | − | | |
| 9396 | 45 | − | + | − | | |
| 944b | 46 | + | − | − | | |
| 951 | 47 | + | − | − | | |
| 953 | 48 | − | − | + | | |
| 954a | 49 | + | − | − | | |
| 968 | 50 | + | − | − | | |
| 971 | 51 | + | − | − | | |
| 983c | 52 | − | + | − | | |
| 985 | 53 | + | − | − | | |
| 990 | 54 | + | − | + | | |
| 998 | 55 | − | − | + | | |
| 316 | 56 | + | − | − | + | − |
| 126c | 57 | − | − | + | | |
| 207-4 | 58 | − | + | − | | |
| 265-3 | 59 | + | − | − | | |
| 29B | 60 | − | − | + | | |
| 305B-25 | 61 | + | − | − | | |
| 326B-39 | 62 | + | − | − | | |
| 34B-11 | 63 | − | − | + | | |

+ indicates differential expression as identified in differential display
− indicates absence in differential display

For transcript number 316, reverse transcription PCR (RT-PCR) was used to detect expression in the breast cancer cell lines.

TABLE 2
Differentially Expressed Metastatic Marker Polynucleotides

| | | | | breast | |
|---|---|---|---|---|---|
| | | | | cancer | |
| | | | | metastatic | breast |
| | | | | to bone | cancer |
| | | | non- | and/or | metastatic |
| TRAN- | | SEQ | metastatic | lung | to lung |
| SCRIPT | | ID | breast | MDA- | MDA-MB- |
| NUMBER | protein | NO: | MCF-7 | MB-231 | 435 |
| 902 | osteopontin | 64 | − | − | + |
| 9112 | nip | 65 | − | + | − |
| 9132 | Ca-dependent | 66 | − | + | − |
| | protease | | | | |
| 9158 | IGF-R | 67 | + | − | − |
| 9174 | ILGF-BP5 | 68 | + | − | − |
| 9177 | lactate | 69 | − | + | + |
| | dehydrogenase | | | | |
| 9202 | ufo TKR | 70 | − | + | − |
| 9210 | eIF2 | 71 | − | + | + |
| 9212 | glutaminyl | 72 | − | − | + |
| | cyclase | | | | |
| 9213 | gpl30 | 73 | − | − | + |
| 9222 | TGFb-II | 74 | − | + | − |
| 9232 | E-cadherin | 75 | + | − | − |
| 9239 | serpin | 76 | − | + | − |
| 9250 | secreted pS2 | 77 | + | − | − |
| 9260 | LIV-1 | 78 | + | − | − |
| 9315 | GTP-binding | 79 | + | − | − |
| | protein | | | | |
| 9317 | vimentin | 80 | − | + | − |
| 938 | cytochrome C | 81 | + | − | − |
| | oxidase | | | | |
| 9382 | Hsp 90 | 82 | − | − | + |
| 9394 | integrin a6 | 83 | − | − | + |
| 956 | desmoplakin | 84 | + | − | − |
| 970 | osteogenic | 85 | + | − | − |
| | protein | | | | |

+ indicates differential expression as identified in differential display
− indicates absence in differential display

Within the scope of the invention are variants of the proteins described above. A variant is a protein encoded by a polynucleotide wherein the global sequence identity of the DNA, as compared to the corresponding SEQ ID NO: herein, is at least 65% as determined by the Smith-Waterman homology search algorithm as implemented in MPSRCH program (Oxford Molecular) using an affine gap search with the following search parameters: gap open penalty of 12, and gap extension penalty of 1. The protein encoded by the DNA having the sequence identity described above will exhibit the percent activity described in the preceding paragraph.

Also within the scope of the invention are fusion proteins comprising the proteins and variants disclosed herein. Proteins preferably used in fusion protein construction include beta-galactosidase, beta-glucuronidase, green fluorescent protein (GFP), autofluorescent proteins including blue fluorescent protein (BFP), glutathione-S-transferase (GST), luciferase, horse radish peroxidase (HRP) and chloramphenicol acetyltransferase (CAT). Additionally, epitope tags are used in fusion protein constructions, including Histidine (His) tags, FLAG tags, influenza hemagglutinin (HA) tags, Myc tags, VSV-G tags, and thioredoxin (Trx) tags. Other fusion constructions can include maltose binding protein (MBP), S-tag, Lex A DNA binding domain (DBD) fusions, GAL4 DNA binding domain fusions, and Herpes simplex virus (HSV) BP16 protein fusions.

These fusions can be made by standard procedures in the art of molecular biology, and many are available as kits from, for example, Promega Corporation (Madison, Wis.); Stratagene (La Jolla, Calif.); Clontech (Mountainview, Calif.); Santa Cruz Biotechnology (Santa Cruz, Calif.); MBL International Corporation (MIC, Watertown, Mass.); and Quantum Biotechnologies (Montreal, Canada).

The proteins of the invention, and variants as described herein, can also be used to detect protein interactions in vivo, using the yeast two-hybrid system, for example as described in U.S. Pat. No. 5,674,739.

In addition to the ribozyme and antisense constructs described above, the polynucleotides of the invention can be used for inhibiting transcription via triple helix formation as disclosed in U.S. Pat. No. 5,674,739.

Those skilled in the art will recognize, or be able to ascertain, using not more than routine experimentation, many equivalents to the specific embodiments of the invention described herein. Such specific embodiments and equivalents are intended to be encompassed by the following claims.

All patents, published patent applications, and publications cited herein are incorporated by reference as if set forth fully herein.

We claim:

1. An isolated and purified human protein comprising an amino acid sequence which is at least 85% identical to an amino acid sequence encoded by a nucleotide sequence selected from the group consisting of SEQ ID NOS: 1-63 or the complement thereof.

2. The isolated and purified human protein of claim 1 wherein the amino acid sequence is at least 95% identical.

3. The isolated and purified human protein of claim 1 wherein the amino acid sequence is encoded by a sequence selected from the group consisting of SEQ ID NOS: 1 -63 or the complement thereof.

4. A fusion protein which comprises a first protein segment and a second protein segment fused to each other by means of a peptide bond, wherein the first protein segment consists of at least six contiguous amino acids selected from an amino acid sequence encoded by a nucleotide sequence selected from the group consisting of SEQ ID NOS: 1-63 or the complement thereof.

5. A preparation of antibodies which specifically bind to a human protein which comprises an amino acid sequence encoded by a nucleotide sequence selected from the group consisting of SEQ ID NOS: 1-63 or the complement thereof.

6. A method for detecting metastatic tumor cells in a tissue sample, comprising the step of:
measuring in said tissue sample an expression product of a gene which comprises a coding sequence selected from the group consisting of SEQ ID NOS: 1, 2, 4, 5, 9, 11, 13, 14, 18, 19, 20, 22, 24, 26, 29, 30, 33, 35, 36, 38-41, 45, 48, 52, 55, 57, 58, 60, 63-66, 69-74, 76, 80, 82, and 83, wherein a tissue sample which expresses the product is categorized as containing metastatic tumor cells.

7. The method of claim 6 wherein the expression product is protein.

8. The method of claim 7 wherein the protein is measured using an antibody which specifically binds to the protein.

9. A method for detecting metastatic tumor cells in a tissue sample, comprising the step of:
measuring in a tissue sample an expression product of a gene which comprises a sequence selected from the group consisting of SEQ ID NOS: 3, 7, 8, 10, 12, 15-17, 21, 23, 25, 28, 31, 34, 37, 42-44, 46, 47, 49-51, 53, 59, 61, 62, 67, 68, 75, 77-79, 81, 84, and 85, wherein a tissue sample which does not express the product is categorized as metastatic.

10. The method of claim 9 wherein the expression product is protein.

11. The method of claim 10 wherein the protein is measured using an antibody which specifically binds to the protein.

12. A method for determining metastatic potential in a tissue sample, comprising the step of:
measuring in a tissue sample an expression product of a gene which comprises a sequence selected from the group consisting of SEQ ID NOS: 1, 2, 4, 5, 9, 11, 13, 14, 18, 19, 20, 22, 24, 26, 29, 30, 33, 35, 36, 38-41, 45, 48, 52, 55, 57, 58, 60, 63-66, 69-74, 76, 80, 82, and 83, wherein a tissue sample which expresses the product is categorized as having metastatic potential.

13. The method of claim 12 wherein the expression product is protein.

14. The method of claim 13 wherein the protein is measured using an antibody which specifically binds to the protein.

15. A method for determining metastatic potential in a tissue sample, comprising the step of:
measuring in a tissue sample an expression product of a gene which comprises a sequence selected from the group consisting of SEQ ID NOS: 3, 7, 8, 10, 12, 15-17, 21, 23, 28, 31, 34, 37, 42-44, 46, 47, 49-51, 53, 59, 61, 62, 67, 68, 75, 77-79, 81, 84, and 85, wherein a tissue sample which does not express the product is categorized as having metastatic potential.

16. The method of claim 15 wherein the expression product is protein.

17. The method of claim 16 wherein the protein is measured using an antibody which specifically binds to the protein.

18. A method of predicting the propensity for metastatic spread of a breast tumor preferentially to bone or lung, comprising the steps of:
measuring in a breast tumor sample an expression product of a gene which comprises a sequence selected from the group consisting of SEQ ID NO: 1, 5, 11, 18, 20, 22, 24, 30, 33, 35, 36, 38, 45, 52, 58, 65, 66, 70, 74, 76, and 80,
wherein a breast tumor sample which expresses the product is categorized as having a propensity to metastasize to bone or lung.

19. A method of predicting propensity for metastatic spread of a breast tumor preferentially to lung, comprising the steps of:
measuring in a breast tumor sample an expression product of a gene which comprises a sequence selected from the group consisting of SEQ ID NOS: 2, 4, 9, 13, 14, 19, 26, 29, 39-41, 48, 55, 57, 60, 63, 64, 72, 73, 82, and 83,
wherein a breast tumor sample which expresses the product is characterized as having a propensity to metastasize to lung.

20. A method of predicting propensity for metastatic spread of a colon tumor, comprising the steps of:
measuring in a colon tumor sample an expression product of a gene which comprises the nucleotide sequence shown in SEQ ID NO: 56,
wherein a colon tumor sample which expresses the product is characterized as having a low propensity to metastasize.


SEQUENCE LISTING (85 sequences)

SEQ ID NO: 1 - DNA, 142 bp, Homo sapien
  misc_feature (1)...(142): n = A,T,C or G
cacccaagaactaagaaacaaagggagaatgtacttttgtagcttagataagcaatgaatcagtaaaggactgatctacttgctccaccacccctcccttaataataacatttactgtnnatttcctgggcctaagactcta

SEQ ID NO: 2 - DNA, 331 bp, Homo sapien
  misc_feature (1)...(331): n = A,T,C or G
cgcgagcagacaacataatttatttccagaaaacaacagaaatgaacatcatcatgaatacatgaaatcggctgtgatgtgtgaactgctaagggccaaatgaacgtttgcagagcagtgggcacaatgtttacaatgtatgtgtatgtcactttcggtacctgtgaatgcatggggacgtgctgaacccgaaaaaaagtgcctttccataaggactgcaatanagagggcaatttaccctggtggtacacggaacctanattcactcctgccatgccttgccaatagtaanctgcagggtggaacaagaaatcacttgctctggggggaa

SEQ ID NO: 3 - DNA, 1112 bp, Homo sapien
  misc_feature (1)...(1112): n = A,T,C or G
ccnnnnnnnnntncntnnnnncnnnnccnnngnnnnnctngcccnnncngctnnnccccnnntnctnntngntnangnncngaanccgcnnnnngnnnnnacnatnntnngncgnnnnnttcgttnnnncntgnntccncnnnnnctngtncnnnnnggnggngcgcnccnccnancctncctcnntgnnncnnnctnntnnctnngctgngtctcncngcncngngcnnnnnggggtctnccgtnctncnnnnncnnngttttangncngnaanacgccgcgncgagcttttagccatgggggataaccgaaccaaacntnacactctcagaggatccacctntgggtgcaagcgaaactngancnatctatactctcganggtncaaggacattgntgagagaaatgganncacagcccacgttcattgggtangagactccnattaanatttctgtctccccngatgggccctagacccatgaatccctattangatcccntcagcggccanacncngtggctccncctgtaatcccccacntcgggaggctgatgagggcgaatccaaggtcaggaaatntatatagacncctggctaaccggngaaccccccctctaaaancaaaaaaaaannccnncnngtntttanagggngttntttttcntcgccncgcccgncncgnccgcttnctngctccncctgnncnnncntccctncnncnntgntcancccngcgnnncgcnncntnccttnntgngtctggtcncncttcnncctctcttncnccnntgtccntngctctcagccnctgccccnccctnnccntnngtgnnnccnccntnatgnccncncnanaggngcangcnntggcncgctgnccnntgtntgtcnctcnacgganantgnactcncnactnngnnacgcnatnnnanctctgctctcagatgacagcancggnntnnncngcctctancnncngnnncnnagccnncgannnaggnanccgcgntcantcnnntttcnctctncnntngcatntctgatngccgtgnctncctcnnttnctcnagcncntnnccacctctcgtttagncnctnnncnnann

SEQ ID NO: 4 - DNA, 183 bp, Homo sapien
aaaactatgaattccatacttgaggtttcccagccaattgctcccttctgctttagaagtgactaggtactgagagtacaaacactcccactttataatgaaggcgtcatgtcaccccttcctttacaggtcctggggtccaggagacccagaatgaaggtgtcagttgggcatgaagtgtta

SEQ ID NO: 5 - DNA, 1092 bp, Homo sapien
  misc_feature (1)...(1092): n = A,T,C or G
ttncagaccaagaagacttgatnagctgaaacccattgcnctacttggaangtgatcngcaaaagctgcctcagtcanacaccggggataaatctggatttgggttccggcgtcaaggtgaanatnatacctantaangaacnctgtacantgccncaagcangtgangaccncccacgagtttacatnaatacaatnctgaaacnacncaggctggttttatatctacatatttgacttaccactatcncantaaagtttngcacctttcnccgaacgaaaanaaccccccntnntgnnttcttttnaaaanaccntngnnccncnttnccgtcncnccccnnatantnnncnnatccccccctctnccnntccntnnncgtaannggcgtngcttntgcngtntntgtcccgttttcctccgcttngtcntttntctatatnggctnntnttatnccnngcccttcgtcncctnnngnttcgtctgtncntagtcctcntnctngagccccanttgntacttcnngcttcnnctccgcattccntctccgcncnnancncnnntctcanannatgnncnntnnctncnnccnatncnccctnanagnttcgnctagaccntcnacnttgtntcccgnnctcttagngntctgctnctagtgtntnnctcatctcctctncttctctctcctttgacncngnncnctccatcntnntctgnctttctcatcncnnnnngcccctnctcncnnagtntgngtgcncnnncttnnnntcnanctngtcgcctccgttttcnactnnnnccnnngcngnncgnnngctctttctntcnnttagactnaccttntctgnnnnntcannctagcnctgtccntctctnntctgcatcnttanacatcttnntcncccnctcgcancntnctnttnacnctcncatacgttnccnnnctcagtccgcagnnnngttncntncngtcntctcgcgnctcnnntcctctctnnnacncncctggtctncgnctcgctccnncccatncntncctcgttgntcnnnntcnnatacgtntncangccncntctctccnctn

SEQ ID NO: 6 - DNA, 504 bp, Homo sapien
  misc_feature (1)...(504): n = A,T,C or G
ctggagcgggatcatttanaatactttacagatatntgcaccaggtacatntatntgcgtccattggtagcacagctgagacctgtgtctcacatcagcctaggtgaagcctactacaaataatgccaagggagaanagccagtacactatatggtttatactctttatccctttattcatagcatgttttttaaaaatgttatattatgcaacagatgtgaggcagcanctaagctatacttaagaattttctctcaccttccaaaccaaagtgtcctgaataagccaggagacttattcttttgtgcaccctggtgcacatctgactgttgtcctanccanaaactctctgaggccactgaaagaacagtggccctatcgatttcattcctaggtctcaaaaatacnatgtngccttgtaacataattagggacagcacctctatttcacaattataatctaaggtaggataagacgacacagcagcaataaacttacaagt

SEQ ID NO: 7 - DNA, 1132 bp, Homo sapien
  misc_feature (1)...(1132): n = A,T,C or G
gcgngccccctngtngnncnttntncncngttttctgctntntttatnngaggnctnggtnnttnntcttagggnnntngtncggtcnngttnntgttncgagcagaaagtgnatatttcatgcngccaagcttntttattgaaaantcctaattntattgnccgtntagtaacatgtttgttcnacaangctaatttctnataaancaaaacacannnttttcttataagtngtataaattatttnatttacagaaacttgtttcaaaacanatgnactanntatttctnctcttttaaatanccanactaattttctatccctngacatctgttcatgttctatncagcagccaacacaaagtccanctgagagctcttgattaangtgtncgnattatctagctacttccnacgttttnggngcnngaaatgncttttaanancctggcctcaaaaaanaaaaananccccccgnnnaggggnnttccntntanaaaaanggntcnctcnnccngtnngagactgtctccctgnntnngnnnntcgctntnatcangngccncnangctcnccntcnctnnngcattngatnnntancnnnctgagatgngnntangctgntncntnngtgtcntangtctcgacgttgnntggntntangnancgncnntntnnncnnattgncgagngnntaagtgtgctcttctcntnacntctntcnnnancntctnngatgttnatacggccgtgcttncttatcnntganancgntctnannanntncgnatgagnntntactgcncncntgtgtcatctttctctctantgtgtnctnnanncnngtnattncgcnnnactgntantnagtggtatnnaganntcgnncgcnngngccnntttnnctgtngnnatnagntntcanganattnatncnntctncgtgataganagntnagtgnnggntctgactgatncgtgtcctagtnncngtgacatcgnncgttanngtcngcactctagtananntnagtnngangntgtanatnnntctcntgtttcagtnnagncccncgagcgcntcanntntnantgtctcntctnngtcgtanncntgtcgagtngtnanann

SEQ ID NO: 8 - DNA, 736 bp, Homo sapien
  misc_feature (1)...(736): n = A,T,C or G
ntgggcccgacgtcgcatgctcccggncgncatggnnnnctggtttggtcanatgtgaataacgnagaantgagaccacnganaagaaccacantgtnanggnncttgcacntgntangaantnagnaatgcctttttnctgagggcnttnggnnntcatnnangggngtgnggnggnttncacctgtaataccaccactttncnatgccactgccngtgnatcaccngnngtaaggacttcaanaccagccttatnaacntgggnaaaccntntntctactaaaaatnctnnaantatctgngcnnngtngngcgttcttntannnccngctgnacnngangncngngnangntantcgcntgaacntgncntgttanagtngcantgagcctaaatcacantgatgtattnncatctgggacgacacgancngacgactcncgtactnaaaaaaaaaancccnttnngggggggtttttnnnggtattanntatanttggagaantttgggtcannngaatattnttacatgaaaaatnaggaataactntatntgtgtacattgggttnnaaanangacantantggnnctaaactnttnggggnggaggggnnattagggnnttaattnggnnncttnnaaanncnnntnnngtatnanaanantntttnnanaagngnantngntttaaancctnaangnttnnntnctnttannttnnaannnnanannn

SEQ ID NO: 9 - DNA, 690 bp, Homo sapien
  misc_feature (1)...(690): n = A,T,C or G
tnnncctggntggtcactcccttctgtcctgttagctcatggtgtaagatgatgtcttgtcagtattactgttttgctaagccgcttcattcatgcctacacaatttttttttaaaagggaactttagttaattaagtgataagggacttaaatatgaattanaatggtgcagaaagagataccttttctggatattttaaagtttaaaggtcantttctcttaatctgattatgtgcacatatgaaaatggcacatcatatacatgtaaaatcaggcagtatncatttattaattactgtatttgacaaaggaaactcttaaattataatgtgaaacctggttttatgaaaccaatgactagtgcancatttcagcatatgcaaaaaaaaaanncctnttggngngctgtttacaaaggaaattgttggatttcacgatggtttcagganaanaaggttttcntcatcnagggtaaacntcccggataaggcntngntttaatntnnttannccnnccnatngntaanngtggaaattaancctctgaanaaaanancccacntnnttngccttgggcttnantctntttggcngnancnaaaggnncttnccaggtntcntgnngggccngnngaannataannaannnggggnncttnggaaaccttncnnnaanantncccncccc

SEQ ID NO: 10 - DNA, 395 bp, Homo sapien
  misc_feature (1)...(395): n = A,T,C or G
tggtatctgacnnaataagaatgcacccatttgtgaggggtaatatttatctcangatttactgtaaatatgtatacacacatacaaaaacccaggcattgttaagagaaaatnatggcccagaggttnaaattatcagacagaacctttaanaataattatgattaatgtgttaaaattctagtggaaaagataaataacatgctcagganattttagcanagagatagaaactatntnngaagctcaaatgaaaatgctaggaaatgaaaagcagtattggaggtgaaagattcctttggcaatttatcaacanactggagatggcanaggcataatcagtantattgaaggcagattactatntattatncaancaaaaaaaaaaaccccct

SEQ ID NO: 11 - DNA, 331 bp, Homo sapien
  misc_feature (1)...(331): n = A,T,C or G
aacgaggccnngaggccaatgaggccaacaagacgatgccggagaccccaactggggactcagacccgcaacctgctcctaaaaaaatgaaaacatctgagtcctcgacnatactagtggntcgctacaggagggaacgtgaaaagaacatctccagaggaactggtgaatgaccacgcccgagagaacagaatcaaccccgaccaaatggaggaggaggaattcatagaaataacgactgaaagacctaaaaagtagcaagaagctacatccctcaaacttcggcaatgaaaataaagtttgagaagctcaaaaaaaaaaancccttttg

SEQ ID NO: 12 - DNA, 693 bp, Homo sapien
  misc_feature (1)...(693): n = A,T,C or G
tncaacgcgttgggagctntnccaaggtggnctagcnncattaatgccctaccgtgggaatatggntgaagatcttgactaggggacttatgaacccatgcagccgtgcccaaatcctaccaaactgaccttactttcttgaagacggaattgtagtatggtcgagctcatgctttttgtagtaggccatncaaattcgattgactggctaaaaaagattgttagtggaggctggaagaaacattttggctgatgatagatgaatagagcttggaacaatcaaaaggaaaagcagaaagtctatacctattcataagaaaaagttagtatgtttaccgaacattatnaaagaattatgacattttcaaagttttaaaattttattttgtagggacggggtctcattgtgtagcccacnctggtctgtttcttgaggatttactatanactgggctgtattcaaagcattggggatacaggcatgaatgagcccccattgcctgaacttaccattcaatctgggcagtgaaagaanagggatgntgggaganccttacaaagatgaaatgtcgctaactggagaaatccctactttcagtcagactgaannggaacaggtagtnactgtgggtagccctctttgggnangggtngattttccacatgtgcccagttaagggccnagaacattaa

SEQ ID NO: 13 - DNA, 305 bp, Homo sapien
  misc_feature (1)...(305): n = A,T,C or G
ttggtatcnggggatgggngaggggagatagncccgaagcatcccnnattctcagtaaactccttggnatcanannatatcntggccnaagaaccncncaccntctntgggttagaaataccgctntatngngtatgaggggatngggcntacgnnataatttnctatngganggtattnccgcactantgacnagttctttctnnggtccatttnnaacnacantnttgacattgntgatctgcaannctgtaaaatagtcttncagtgggcaatnnntgcacaactgggttnggtntcanaca

SEQ ID NO: 14 - DNA, 308 bp, Homo sapien
  misc_feature (1)...(308): n = A,T,C or G
agcagacaacntaatccaagccatttaccaaataantatatgcgatgcacattgaatcctggcgctctagatatantgccccaaaggaaagagnacaaagtnttccncccntagttctacnatgnctatccnctatcacctnctgnttcnnaagntttntaaaaataaattctcttgtatancatccnatatcncaccggtccaaagcgcaacaatctgcaattcanaanttccaacaatcnatntatgnactttcntaggtccggtgttctaanatntaatattctaacacttactctcagatctta

SEQ ID NO: 15 - DNA, 304 bp, Homo sapien
  misc_feature (1)...(304): n = A,T,C or G
ngtnaagggatatttattcctgttttaaaaggatacaaccaaggtagggaaggcttcgttattggtgattattcagaagacctattttctttacatatgctatggaaacaatactgttttccgctacagaatacagtttatgattatacttttgtaaattgcctgcttttcccctgtcatctgctaattccaatttgatactgttctgtgttcaaaaatacagcatgagcaagctgtaatggtgcctgtcgagagtcccagctgcttggggggctaaggtgggaggatcatttgagcccaggag

SEQ ID NO: 16 - DNA, 703 bp, Homo sapien
  misc_feature (1)...(703): n = A,T,C or G
ccggtngnctaaaaaggaccagcctaatgtagaaggtgggtatttggaccagaggctttagattattattttagatcctacatatacttttatcagtagaatgatttcattnagatgtataatgaaaaagggtaatgcaaaaattatgtaatagataccaaattagggaagtttggcaatttcaatggcatatttttagtcaaggnacacagatggcagtgccataagcaagtctataaatatcggctgcagccatccccctcattttaaatgttgccctaataatcaatgcagttaacaagtatattggctgtgtgtcatgaaatagttcatgttcagatggaaatgttaggttactgtatggtttatggagattaatgaaaatgaatgcccaaaaaaaaaannccntttngnggngggtttnnnangnacngggctggattcaaancattggggatncangnttnaatgngnccccatttgnctnaacttaccttnnannntgggcnntnnatngaanangggatnntgggannaacctttnnangntnnaantgtnnncttactggngnaaannnccntaanntttnnnnntnnnnnngnaanggggnaannnnnnnntnancttntgggggagncnnttntggggnanggggggntnnttnnnncntnnnggccnnnnnnggggcnnnaaantttttgn

SEQ ID NO: 17 - DNA, 171 bp, Homo sapien
  misc_feature (1)...(171): n = A,T,C or G
tccgcntctaagtaattcatcaataacgcatgtccacttaatgtgaaaattggtaccatctaatanaatcttcaacatggcnatccacnctattccaataatgaaatgcaaatttccctgccttctttactanggtcatttntagattcttgaggaatgagttctactctt

SEQ ID NO: 18 - DNA, 689 bp, Homo sapien
  misc_feature (1)...(689): n = A,T,C or G
antnngcttnggtactaagcagaatcacttncttgggaactccatgtaactngtggcttttgtgattgaaatagcatcagtaaangtctgaccctgtggtaaagacacatatgngcgtggaccnggctatgtctgactttgtgctgctcaggacactctctgtnaccaaaagngagagancctggannacctcanggggtcanatgtttgaaggagctgctgagtatcctggcaggcancanagccttaccatcagtttgctgcatggaaggctgtgtgcctctatttccctgctatttgttgaactcccttgagctccggtccttcctaagtgagagagatgatcccaatagcnccaacctgagagggctggggagatgttngaaggaaagcttggctggggagctgaatctggcctgtggtacatgcttggtaactggtggccagganacccgggngtgtgtnctgggactgtcncactctgctgaccagggtattgaaagtccccnctcaaanacacagaatntntctgaccaagggtangtatganatgacntgtggagcactttgnataaactggttctcatnggnggtccccttgaanaggtgctnnatctgttcaaaaatacgtggctgagctntanacccngnatcctctgtcagagacatgggcagggggactcaatgct

SEQ ID NO: 19 - DNA, 721 bp, Homo sapien
  misc_feature (1)...(721): n = A,T,C or G
tatanatactnngctatgctttctaccctgtgtgcctggagacctactatggaaaaangatcagccaccttaccttctactgggtacctgctgtgagtctgcctatgccacaacgattaatgangggagggtacccaagngacaaanccnacatgccgcttacagcccccgttggatngntgctcattcaacagtcttgcattcagtaggtgtttgacatcacctactatgtgncaggctctatgctangnactggggatacaggagagantnaagcgtaaagtctttggtctcaaggaatttgcattctagaaagtctaagatgtaataaatgtactgtgggacatgttaaataagtgctataacgaaatataaagggtttgggagcaaaaaanaaacccnnttgtggggntctntnccnctctgatgaagcttacttacttttaaccttnccttctcctttaaaggtgtttcctggttcccctttcctttacagattggttattggtcttgctgaggagtaggactacaattnccagcattctnctggaagccaaagctgtgctacaattgnnccaaagaagatngtaatcttaagcgcccntaatggtaaaatngtattaaaangtggacctttgacaaataaattgnttcgatttcngaattccgggttngnagcttngngntnccaaaaacccttnggggntcccttttgggcacc

SEQ ID NO: 20 - DNA, 248 bp, Homo sapien
  misc_feature (1)...(248): n = A,T,C or G
cttaaacaccccncccatctncnccccagaatgagntaancatactcntcnntactgnatctccgtatccgtccctacncnggnttgtgaggtgtcattagcngatattactcctcatcnncatcntgancannatcccccatcnnccatatgntgatnannacaaaccatnctattncgccgnngaagccnntcnnttcattggattcntagaccgcanangtcctnattcngacacngaatcggta

SEQ ID NO: 21 - DNA, 298 bp, Homo sapien
  misc_feature (1)...(298): n = A,T,C or G
ggtctaagggatgtgatgngagcatagaatttanctntatggncatantagggacatntgctgatntacntggnctgcggtcnntgaaaggtggngnatgatgactgatgtcatnagtagtacnanggactncgnnanctgggatcngggnttacnttgttcatngtnagagtgnnancnaagtanatgntaggnataaagatgttncgggagatgggtctacaaantcttttnaagatgntcatcttgaanannatcaagtgtgnttggtataatgactatcattatacaatgtcaa

SEQ ID NO: 22 - DNA, 591 bp, Homo sapien
  misc_feature (1)...(591): n = A,T,C or G
tcgctagantactattcggccgcaacggggagcctgatgaggacgcttatgatatgaggaaagcactttccagggatactgagaagaaatccatcataccattacctcatcctgtgaggcctgaagacattgaataaccctgggcagtggttcttaggcagatactctagatgctttatggacaatattattttcattggatgattctggagctctattaggagaaaagtaatcattttaggtcttaaagacttcaagaaaatacaggttatcaatttattttaaatctcattgtttccagttagcaatatcatacctattaaagctgttcattgtaacaaaattcaatcaaaaaggcagctaggtcagaaggaaacataccactctcatggttcatagtattcactgtatgtatgctagggaaaagacttgctccagtctcctcctcagttctgtgcctgagaaccactgctgcatatatttgtttttaaattttgtattgaactgttaattgaagctttaaaagcatatatgaaatgtataaatctaagatgtataatacattattgactccaaaaaaaaaaacccctt

SEQ ID NO: 23 - DNA, 755 bp, Homo sapien
  misc_feature (1)...(755): n = A,T,C or G
gnnnnnngttnnnnagcnggttnggtncngactcccntttatnatgagggacactgaggcttcaagagattaggagacttgttcaaagacacacagctggtaagtgatggaggcaggatttaaacctgggtttcactgcatttcccatcactggcttttagccatgatgctctactgtgtaaccctcttaattcttgacctgtggctataaagtatgtattgagagacaggccctccctgagataactttccagccttgacaaaggcacacccttggttcattccttggagtgtaggacctagattgtgacaagcccagatgagtgtgtctggcagaggggagcagatctgaggccaccatatgtgttcacctagccctaaggagtgccagcttcgctggtatttgtacagcttccatcaggactgctcattggccacgttctttcctctccctgccacgttgattaatactcacataaattaatgctcacattagtgttcaagtatgcaaatgagtgcttaaaatcatcactcacacaatgaccagactgaggatataacacacaagagcccctctcctggtaaccccacaatcatgcagatgtgttgacttctctgcattaccagtctggtaggcagggggatatgacagttagaaacagtctttcanacagcagttctcaacaccaggtcccttgctgcacaatcgaatcacctgggggtttaaaaaaatatcatgccagtcagccacnntt

SEQ ID NO: 24 - DNA, 513 bp, Homo sapien
  misc_feature (1)...(513): n = A,T,C or G
ctttctacccaacaagcatagaatatacattgtatacatcagaaacacgggacattctccaaaatagaccatatgatagggcacaaaacaagtctcagtaaatttaagaaaatcagaattatatcaagtactctctcagaccacagtggaataaaattggaaattaattccgaaaggaacactcaaaagcatgcaaatacatggtaattaaataacctactcctgaatgattgttgggtcnacaatgatatcaagagggaaatttaaaaattctttgaactgaacgataatagtgacacagcctatcaaaaactctgggatacagcaaaagtggaggtaagaagaaaattcatagcattaaatgcctatatcaaaaatctgaaagagcacaaataaacaatctaaggtcaccctcncagaattggagaaactagaacagtccaaatccaaacccngcagaagaaaagaaataaccaaatccgaacaaaactaaatgaattgaaaaaaatcccc

SEQ ID NO: 25 - DNA, 574 bp, Homo sapien
  misc_feature (1)...(574): n = A,T,C or G
cgatccaagagattagaancccntggagtggagcatgcttcnctanaatnccacctgatncttggctnaanacantnngctctantttgctttgtgcccgtccacacaanctaaaaacaagggatggggggaccncnagtgtctaatatncntaatatccntccncnggcaaatgaatactttttacacacttgtannttntggaggganggggtnatnatgaggggaangggaaaggatgaggagaaatccaggatnanangtctcttcgtcctctcnagactncctcacactctntgtggtnaccngggttcgttntgtccaatggcagacattatactccatantctacccnggcttnntcgggttgggacgccannactcccccnagtngtnncccccnancagcnatacacaagtntgaacgggttttgtggccantcatcgcaatgaccttntcctcnactcnaagaaaantaaaccccttcccccngattggtttctaaatctttcaccccatctaaaatagaaagcnctnagtgggangggttnatccccccnttaccnttaaaac

SEQ ID NO: 26 - DNA, 185 bp, Homo sapien
  misc_feature (1)...(185): n = A,T,C or G
gnacnattggcaatgacngaaagaatttgaangatgnacaagtnaaagnnacagtggcaaagaatcttcngggcgcgtcaaaacaattgggtgnattaaggacaanctcggtcancagtataanctctctttcncgnggattantngncataatcatnattctgacnngtaggacattnccaacc

SEQ ID NO: 27 - DNA, 270 bp, Homo sapien
  misc_feature (1)...(270): n = A,T,C or G
ttctggggctctatacaggctcctatttngatccangcgtgctgatgagtgcacagcacgatcacatctggaaaccaccantaccaccaccactacgcacntcaccaaaactgtganagggggcatttcagagacaanaattgaaaancgaatagtcntcacgggggnatgcanacattgaccatgaccaggcgctggctcaggcagntaaagaggccanagatcaacaccctgacatgtcngtgaccagagtggtggtccttacanaga

SEQ ID NO: 28 - DNA, 758 bp, Homo sapien
  misc_feature (1)...(758): n = A,T,C or G
tgctaggtanaaagttacctctaagggaagctctgcagaagaaatcagtgaaatactctgaaagccgcaattacaatcaagaggaacctacttccctcctggcaaagaaacccaaggaaggcgagcggaagatttacttggcaattgaaagtgccaatgaactggctgtgcagaaagcaaaggcagaaatcaccaggctcataaaagaagagctgatccggctgcaaaattcataccaaccaacaaataaaggaagatacaaagtcttatagacatccggaaaaaagatttttacctgtgctggtctatgatgtatgtggcagttgctgtctgcagtttacaatgtattgtnaatgaagattttttaaattctatcttgctgattttttttaaatataanaaactggtacttggtaaagaaatctgtccgtaattnccccccaatcagtccaactatatttaaagccacctgttttcnaattttgatntcctttaatgttnactccaatatccatattttaaatgtcccggataatatcccaaaggtttaaaaaatggaaatntttgaacttcnnttgaananaataaattcccatcctttangggntntccccttncccgttcttccaagaaatgtgaccttccccaaaaaagntnatccctanctttttgnttcccccctgantttctgancccggacantnacgggtttaaaantttttaaattttccaanncaaaaaaccntntnnttttttna

SEQ ID NO: 29 - DNA, 577 bp, Homo sapien
  misc_feature (1)...(577): n = A,T,C or G
ctgctaggtantaanattatggatccacattgtnctgagganacgaanatacttgctgctgatngaggtgaaaacgatattgatccntctggggttttacggtgtgcactgggtgctgcacnnacttgtcaaggtttgntacgtcctctgggcatctgcaaaaggccctgctctctggagtgttgtatgtagtgtaccaaaanagtatttatacatcccaccaatcaaaacacagctttnttacctcatgcgaactcatncaaaccaatagaatntcaacatgttctgtaccttanagtgctcacttactacctctgaacnatactcacgctgtnntttgtctcttncttatctttttgcntcttgtaattaactctttgtttcccttcatcaaatgtaatgtanatcgtgatctattaaaanaaaaatcanggttgcacttgctactttnaanaaaccgantgtggaaacattgggtctnaattcacacaggatcngtanaactgttgtggatactgagaaacntttgaatgttcctccccttattaccatcccgcaaaaaaacccctntnntttt

SEQ ID NO: 30 - DNA, 449 bp, Homo sapien
  misc_feature (1)...(449): n = A,T,C or G
tttacccaataanntataggcgatagaattgatacctggcgcaatagatatagtaccgcaaggganagatgaaaaattataacnaagcataatatagcaaggactaacccctatnccttntgcataatgaattaactagaaataactttgcaaggagagccaaagctaanaccnccgaaaccagacgagctacctangaacagctaaaagagcacacccgtctatgtagcanaatagtgggaagatttataggtagaggcgacaaacctaccgagcctggtgatagctggttgtccaagatagaatcttagttcaactttaaatttgcccacanaaccctataaatccccttgtaaatttaactgttagtccaaagaggaacagctctttggacactaggaaaaaaccttgtagagagagtcataaaaaaaanccctntngggnnnngn

SEQ ID NO: 31 - DNA, 500 bp, Homo sapien
  misc_feature (1)...(500): n = A,T,C or G
tcntggacccnggtccccnngngancaaanaagaagggcnngnttncatngaaaancctgtgattntcgccccggtncaggtgttnanntatggcccncncncatctggtatacgccnaaacaatntanttttacaatnngtnccccancaaacaangttcgtngnnttnactaggtagttaatcccnccccatgttcaaataaagggcccgcgntncnaataaggaanccnccccgantggggtccccgaggccctctccttcataaaanncattcaacttccctcccnctannaaagnaattnttcnaatttttnaaacactccctgtccanggggactttncccccantanctgaaaaaatngcntgacgttccccttcggcctaagggcncaacttanttnncccccaanacccgngggnnaggnnnaaactcccctngaagggaacnactcgcntaaaaanggaataatcncccccnaattattccctncccggg

SEQ ID NO: 32 - DNA, 426 bp, Homo sapien
  misc_feature (1)...(426): n = A,T,C or G
gtctatgatcacatctgacgctattcctatccccttcctccccgggaccttttccccttcctccctgggaccttttccccttcctgtttaanaanccagggctgcctggaggaagctttgtcagatctagtggaatgtgacctccctggaatatgtgcccaggggtttgtctaagcagtttcaggctatggcctttactccatctggtccccatccctcttatctctctcatgtgtggctgcacctggacgcttggaccatagctgtcacagccccctggggaggaacccactccttggccatntcagcctgtgcaatgcaaggctcttgtttgatctgtgtgctgacanaaagcccagcttccttaagaacttttcatgtggaacactttggttttganaagaaaataaatcanaaaccattaaa

SEQ ID NO: 33 - DNA, 375 bp, Homo sapien
  misc_feature (1)...(375): n = A,T,C or G
ngttgcacctattggccngctggtctcgactcctgacctcgttatctgcctgcctcggcctcctaaagtgctgggattacaggagtgagccacagtgcctggcctgtcaagacttctcttaagttaacttcctgagaagtgatgtctaaaagtatctttgctggtgtgagaactccagtttccaacacatattatttccctcaactatttggaatattttagaattttaattccaaaggattagtttgaatacaagtatgccacataactcagttttcgccatcttncatttcttaacagtgtaaattaaaagctaataatcataataataaagtgcatttaattatcttcgaaaaaaaaaaancccttttgggg

SEQ ID NO: 34 - DNA, 809 bp, Homo sapien
  misc_feature (1)...(809): n = A,T,C or G
ttgcacatgctggccaggatggtctcgatctcctgacctcgtgatctgcccgcctcggcctcccaaagtgctggaactacaggtgtgagccaccacgcctggcagctttgtgttcttttctttctgtgatcttgccttagatcacacagataaaacatgacaggacctggaccttaacacagtttggctctcaatcctgttctcataaccacnactgccttcatttatctgtgtcatcctcagacctgacacatagtaggtgctcagtcagtgttcactaagtaaatgatgaccaagaactctttgactgggtccaaggtgcttatcccaatacttcgccatggctacctccctcattcctcagctgacttgctctctctagcctggctgctcctattttatttcctaaacatggacccatggcaataagtttaaanctaacangttgatacggtacccatccataatttaatnaattntggggctcatgcaaccncaaaaaccagaacccaaaactacctgtncncaaacaacaatcattttnggtngggatcccntnctngcttggnccctttttttaaaatgtccattccccccggactttaagaaattgaaggaatncccggaaantattgttancgggcccccttnagngaaaaaggtggcnctccnnncgggggccctccctgtccctgaaatttnaaaacccccctcccnntttaaancccttaatcccggntaacancnaaanaaaattctagggcccaaacccannggtttggttttaaaaaaccntntatttttttnat

SEQ ID NO: 35 - DNA, 192 bp, Homo sapien
  misc_feature (1)...(192): n = A,T,C or G
caccttattgggatacagcagtgaattaagctattaaaataagataatgattgcttttataccttcagtagagaaaagtctttgcatataaagtaatgtttaaaaaacatgtattgaacacgacattgtatgaagcacaataaagattctgaagccaaaaaaaaaaaccccaanggggntnnttttnaaaaa

SEQ ID NO: 36 - DNA, 368 bp, Homo sapien
  misc_feature (1)...(368): n = A,T,C or G
ctgctagtaccaantattatttaagantacttttcactactcctaaataatgacacagatacgtttgtcttacacatttcactttattgtcaagttattagtatgtttattttcaaaagttattttttgcaatttctttttattattccgtactttttaaatttacttcattatcacgtcttcctttattctttttaaatagtttttgcttttgttattttgttttcccttttttactcttggtttgtaatacctctttccttatttgctcctttctcatttgatctcaatgttaatccaactgttttccacatctgattcactaaaattttagcccaaaaaaaaaancccntttngggggngntttt

SEQ ID NO: 37 - DNA, 219 bp, Homo sapien
  misc_feature (1)...(219): n = A,T,C or G
ggccccatttcactctccatantggcncttnctngaacaggcgtnctggatnagtgcacatacnatcccatcnacntgcacctatancncttccactacgcacatcaccaaanctgtgaaagggggcntntcnttagacacacaattgcagaatngacnncncancccgggggannctcnangttcaccntgnagcaggngctggctcangctnttata

SEQ ID NO: 38 - DNA, 198 bp, Homo sapien
  misc_feature (1)...(198): n = A,T,C or G
tcgatacagggncagatctgggagccagggcgttgctgatgagttgcacagacgatcacatctgaaaccaccagtaccaccaccactacgcacatcaccaaagcgctggctcnggcaattaangaggccaaagagcancaccctgacatgtcngtgaccnttgtantggtccntaangacacngacatcgcctccaca

SEQ ID NO: 39 - DNA, 560 bp, Homo sapien
  misc_feature (1)...(560): n = A,T,C or G
tttnnatcngnacagctagtcctntaaantaatgacttcatagaaatggcattataatttttaagttgatactctacaggtagctattgatataattagttttaataaaacatgctgcaaccatggtatacaacaaaaatacatttctttggtgattgaaattaaggccgtatttacaatgacttaatataagactgacttttatcctgcttcataacttgtatggagaactcaccaagaaagaattcaatactgtgaaatatgcagcaagaagattggtctttacctaggctgtgtttcctaagctctgagttttcagcaccagtagatttgtattaaaagaaaaaaaaatggggccttagcttctggcttttaattttgccagctaaggacataaaacaaaantaancaancaaaancaaatagccatntgctatcagcatcattatgtaaaagaaaatntattttagcccctaaaattaggaagaatgtaatctcagaataaaggttgtcatttaagttgaataaatatntagctttcgaaaaaaaaaanccccttt

SEQ ID NO: 40 - DNA, 421 bp, Homo sapien
  misc_feature (1)...(421): n = A,T,C or G
atacagggcagcgtgttaggtgaccacaccaggagcctcagcctcggtccttctcagccgtcgggataagatccaggcatgncttttaaatctcagaggtagcagtaaacttttcantnttgcngttagcaagtgtgtgtttgccaataaanccccattatactaatgtgcctanttaatgttcagggaanatctgcttccactgtgtnccnaggggtgncatgaactntgtgagnagccccncnnctggagggatgaatgctgngttaactacngctatcacggatngtgtgntgtgaanaatacatcnacatnaatnttanntgctctgnaanttcccttnttatntgtcaagtaactntttgtaaaantnntnctcccaanttattacngtgattactaatnnattngtnccatgttt

SEQ ID NO: 41 - DNA, 411 bp, Homo sapien
  misc_feature (1)...(411): n = A,T,C or G
aggtagaggttgtgcatgttgtcctttttatctgatctgtgattaaagcagtaatattttaagatggactgggaaaaacatcaactcctgaagttagaaataagaatggtttgtaaaatccacagctatatcctgatgctggatggtattaatcttgtgtagtcttcaactggttagtgtgaaatagttctgccacctctgacgcaccactgccaatgctgtacgtactgcatttgccccttgagccaggtggatgtttaccgtgtgttatataacttcctggctccttcactgaacatgcctantccaacattttttcccagtggagtcncatcctgggatccagtgtataaatcccaattatcatgtcttgtgcataaattcttcccaaaagggatctntaattttttg

SEQ ID NO: 42 - DNA, 408 bp, Homo sapien
  misc_feature (1)...(408): n = A,T,C or G
ggctcccctccctaactctctaagtacttcccttacccactcagtgtggtgatggcacctccctgaatctcctgacaaatgcgaacaggaactcctattcatcaggagccaacttgataactganaagattcctctctcatttatcagcctttgattatctttttgtgtctcttactatttgcgcttagcgagaaaaataaagaggtttgaacaattaagaagtaacaaagagctcatagttcacaaagagcaantcaaaggatgtctggaatatttgaacatacaactgcctttggcatgaggtggcctacatacattctcaggggcaggataggctggnanagctgatcaagctgccgggaaagctgaagcaaaggcagggttggntggaaatcaaaatntctctt

SEQ ID NO: 43 - DNA, 275 bp, Homo sapien
  misc_feature (1)...(275): n = A,T,C or G
tccctaactctctaagtacttcccttacccactcagtgtggtgatggcacctccctgaatctcctgacaaatgcgaacaggaactcctattcatcagagccaacttgataactgagaagattcctctctcatttatcagcctttgattatctttttgtgtctcttactatttgcgcttagcaagaaaaataaagaggtttgaacaantaagaagtancnnggagctcntagttcanaagnagcaagtcaaaggatgtctggangatttgaagggt

SEQ ID NO: 44 - DNA, 246 bp, Homo sapien
  misc_feature (1)...(246): n = A,T,C or G
tttggtcccaagcacatttcacaaangagaatttacacctagcacagctggtgccanganatntcctanggacatggccacctgggtccactccagcgacagacccctgacaagagcaggtctctggaggctnantngcatggggcctantntcntcaatcnaatgagccccnantgctactgcgccccgggggctcccacggcctgggcnnctttcntgcaactgnaaaaggatagnggtatttc

SEQ ID NO: 45 - DNA, 345 bp, Homo sapien
  misc_feature (1)...(345): n = A,T,C or G
tttggctccgtgggacgttgtantgtgcncagacatttccaagggaaattctaaacagtcaccctncccttttgcattcccccaaatcttaagtgtatacataaaaccctgggtacatattgtngtggtaatagaagggaattggnnaaacngtacacttgttatatggaantnactgtggccacctacaaaagacaagttaacaaactgtcntggaggctgtngntgcccanccagggccgctgcnttttgacaacattcccaccctggccactcagcacanttcatggcaggtcatgtctntncactganacntttntganactttttcatatagcanaatcc

SEQ ID NO: 46 - DNA, 969 bp, Homo sapien
  misc_feature (1)...(969): n = A,T,C or G
aattgcagttctttcttgcctttaacaacattagggcctttagaatgagtacctggtgctgtccttccaactctgtgattctctgattccatcctcatttttcaccatcactggtgtactggcaagaaccantatgagatttgaggaaaaatacttggattactcttttttaaaaaaaattatttagatataattcccataccatacaattaacctttttatgtgtataattcagtatttntagtatatccacaaagttgtgctaccatcaccactatccgattccagagcttgtcatcatacaaaaaaaaaaaccccanagtnanttcctttcaaaacnctttnngttnttcnttntncccntgtngcntctagnncngngggntnncttttgtcnntntcnccctncnctcatcntnncnggtctctgctcngngnnncgntntgncttnnantcgctgctnntcntgtattccccgcnctngtnnngtctgcnncgtagccagtggncctcctgntnccnncngnttctntntncggcacanntccanccanctgccatnagtnanannatctctnttcnncanctgntnncagnnttgtcntcntctccgtnccnccngcngctnnctcnttncgcnctggnngncantcgtacctggcttttatccccctntccnnctnttctngatggnntctcntctcnacacctgncgttacgnntctcntntnncnnnanncgttnctntntnncttnccgncngccatctnagctcannctggngcgantcncgctctgngtatcagtcatntanagannngngnntgttnccnncgcgnnntgaganncccncccncttcgcatnacgtangtgnctttntnnatctgctcgtcgtctcnctcatatccnccatgctgncatganactccntantctnncgcnnttctnncgttccctctgcccttnn

SEQ ID NO: 47 - DNA, 361 bp, Homo sapien
  misc_feature (1)...(361): n = A,T,C or G
ggccactaagcaggtcttaccnaatttaagaanattgaantcctatcaagtatctcttctgaccacaatggtatgaaactagaaatcagtaacaggaggaaaattggaagattcacaaatntgtggaanttaatcaacncatgagcaactantgagtcnaagancanatcaaaaggganntcaaaaactctcttgaggtggatgagaatgganatacaacataccngaactcatgggatgtatcacaagcngtgctaagggggaagtttaagtnctagatgtctanattangaaagggaaagatctcanatanacnacccagcnttncncctcgaanaactagaaaaactaagaaaaaact

SEQ ID NO: 48 - DNA, 364 bp, Homo sapien
  misc_feature (1)...(364): n = A,T,C or G
atgatgaccacatntagatggcacatngatgaggactttaatctttccttaaanacaataatgtgttcttttttcttttantcacatgatttctaagtanattttncatgcaggacactttttcaaccttgatgtacantgactgtgtaaaatttntctttcagtggcaacctctataatctttannatatggtgagcatctngtctgtttagaangggatatgacaataaatctatcagatggaaaatcctgttacaaagtataaaagctttagtaatttactcagtgtggtggttttatcctttttgctttttctcccttggtctataatgaaattgttacagcagtgcaaaataaaatcct

SEQ ID NO: 49 - DNA, 703 bp, Homo sapien
  misc_feature (1)...(703): n = A,T,C or G
atggggaatcaaacaatgttaaaaggctantaatacttataggttttatgattcaatttactatgtgtttaaaattgttttttgaaaaaattgagttatgtcnctaaaactgagtctntacagctcaaaaatgaagaaatacntatctccgataagcatattatgtgaatttcaacatcnctattgagaaaaggaatataaatttgaatgaaaatgaaactctatctttctatatcacattgcataggtgtaggctagtgagtactttgatgtaaattgctgtatcttttgaggcntcnatttggcnatatagatcagaattttaaatcngcatactttgtttgccagaaatctatcaggaccacttgtantnattttgttnaaaggaatatcnaacncttggatgttcancncagtattgattgttttanaagaaggaaanggagaaagggaggagaatggaagananaaanggagggaggaanattggaaccnttgacatntgtgatagcatnggatttgctnaacacnctatantatacccctngcatggganaagcatgcacnctnaaacaaggacnngttngatggntctacnntttgacntcagatnnaantaaatnaaaaaaaaaancccccncctctttgnnttcctntcnncgnnnnanncntctccccncnncgnccnncncccgccaccntn

SEQ ID NO: 50 - DNA, 413 bp, Homo sapien
  misc_feature (1)...(413): n = A,T,C or G
tcttggctggttgagtattcaanaatcaggcacggagaagtggggtggatgcaaaccaactgaccactgtggcaccaccagcagtttcagttttcatcttgantgtcnagaggaaatatctaatcttacaactcnttaggggcctggctcagtggctcataccttgtnttcccancactttgggangccgangcnggcntatcacccgcangtcaggattttgagaccaccctggccaacntggtgaaaccccatctctactantcaatacaaancttagctangcgtgatggcatgcacctctaatcccacttacttgggangctgaggcagcganaatcacttgtaacccggaaggcanacgttgcatntgagccaagatcgtgccactgcactccatcctgggctttcta

SEQ ID NO: 51 - DNA, 252 bp, Homo sapien
  misc_feature (1)...(252): n = A,T,C or G
gttacagacaaggnttntagaatatcttatgttttatgctctgtaagttcaaagaagntagcagaaaacataagcatactgaaaagagaaacagaagctattttttaaatacctatgtgaaatctctctatntgaaacaaaaaatacactggatggattagacactgcagaaggaaaatttggtgaacttgagatcttataaataaaaattatccaaaatgaagtgtagagtgaaaaaaaaaaancccctat

SEQ ID NO: 52 - DNA, 875 bp, Homo sapien
  misc_feature (1)...(875): n = A,T,C or G
agaaacgagaatgganattcaaatacgtcngccgggcttggtggattagacctgtaacccnaacactttgggaggnctaggtgggcggatcaccngaggtcnngagtacgggaacancctggcaaaaaccccntctttantctgngaaaacncaactctactaaaanaactactcttagatnggcgtngntgcgcctgcctgttntcccagatacnntttnaggctgangtggggataantnctttaacatgggaagtggaagttgcactgatccaatgtctccacactgcantccagcctgggttanggaatgagaccccncncacggaaaggacaataaaaanccccnnnggnnttnntttttaanggcctcttgntcnttttcttntantgcncgcctncgcnnncnttgntntgtcgantcnnntgcnnttntttcttcnncctcnancctgcttctnntcnnttcgccnntnnacngcttcccccntnctctagcacttnnnttctntcgntccnnnatctccnncttntctnnnccgctcgcgtnnnccntnanctcgnntcntnccctttcttcncngcnncnntttcgncnagatcgtncgnctctatctacttctntccnngntntanatatngatnttacattntgctcnatnacccatnannncntctatgtttatannngtnnnnccnttcaacnnnncnttatgagntcttnactcagctctncgttgntnttccnactanngttgnncntncatgtnctgtcncgtancnctctnctcntcncngtcntgagacnaatctctatntatngnttatncctgcntnctganctncaccgngatctcggcnntntcttctcaag

SEQ ID NO: 53 - DNA, 182 bp, Homo sapien
ccagaagaagggctacatatggactcatgttgggcctactcctgcaataacaattaaggaatcagttgccaaccatttgtagttcacaaattaaaactgggtttccaggcctggtgtggtggctcacgcctgtagccccagctattgcaccactgctctccaagctgggcaatggagtcaga

SEQ ID NO: 54 - DNA, 329 bp, Homo sapien
catgatgcgagactggacatctctcctaccccatgtacacttcagctgagcaggcagaattagagagtcaggactagaagttcagtctagggatcaaataataatagtagctaatgtttaaaggtacctaagatccgccaggagacatactcagtatagttccgtggtttgccacatttcatcttatccagtagcacaggtgaaatttgtcttatgtgtatactgaggaaaaacaagtccctctgataccagcagccaataaatgacaaagctgggatagaaacttacttcattctaacccgagagtccctgttcttgcatggggcaca

SEQ ID NO: 55 - DNA, 312 bp, Homo sapien
  misc_feature (1)...(312): n = A,T,C or G
actcaactcgtttgagctataggaatnggccattcgnngtggctcanacctgtaatcccagnatttnggganacctcactaggatcacntgaggtcaggagttcaagaccagcctgtccaacatggngaaaccccatctctantanaaaatacagaaattatccaggtgtggtggctggcacctgtaatcccagctacttgggaggccaaggcatggaaaattgtctgaacctgggaagtggaggttgcggtnanctganatcatgccattgctctccagcctcggccacanatcaagaccctatctcaaaa

SEQ ID NO: 56 - DNA, 565 bp, Homo sapien
acaatttcacacaggaaacagctatgacatgattacgaatttaatacgactcactatagggaatttggccctcgaggccaagaattcggcacgaggggatccaacgtcgctccagctgctcttgacgactccacagataccccgaagccatggcaagcaagggcttgcaggacctgaagcaacaggtggaggggaccgcccaggaagccgtgtcagcggccggagcggcagctcagcaagtggtggaccaggccacagaggcggggcagaaagccatggaccagctggccaagaccacccaggaaaccatcgacaagactgctaaccaggcctctgacaccttctctgggattgggaaaaaattcggcctcctgaaatgacagcagggagacttgggtcggcctcctgaaatgacagcagggagacttgggtgaccccccttccaggcgccatttagcacagcctggccctgatctccgggcagccaccacctcctcggtctgccccctcattaaaattcacgttcccaaaaaaaaaaaaaaaaaaaaagatgcggccgcaagct

SEQ ID NO: 57 - DNA, 798 bp, Homo sapien
ggaacaagtagaagggaagagggaaatggagagcatccttatgactttacaaagggtggaaatgaggatggagggatacagaagtctgcacagctgtaaaggttttatagatgtctttgccttcccttctgaggaagggaagaagtaatgaaagcacatgtgaataaccccttccatcccattcacagcatcgcactcccagtccttaaggcaaagggaggcagtgctgaagcattggtggtgcagtgtaaagagacaagacctgatcatctgatcacacttgtgccaacttgattcatattgggcattactaacaacccctggtcaaggtaaataggttgaacaatcaataacattatccctgcctgcatacatgtgaacaaaagctatagaggacatgcaaattctacagtcattcctcatatgctttagacagagtgcagctactggaatcttccagatttcagtgttttaaaatcagagctctgaatacacaaaaggaaagagaaatggagcagctgacatattttaagctcacagtgatactcagtgacaggagcacagagctctaatgtccacaggatgttgtagggtagggtctctcagtaaatcaagtcccttacctatgttctgacactgaggctcttggagctatgggttagaaatccaggaggcaatatgtctttattctaatgaagtcctcatcttgcactcagaggcccactagtttgcccttctatatattaagtaaaaccaagagaaattaaaaaaaaaaaagccctatagtgagtcgtatta

SEQ ID NO: 58 - DNA, 729 bp, Homo sapien
aagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgaattgtaatacgactcactatagggcgaattgggccctctagatgcatgctcgagcggccgccagtgtgatggatatctgcagaattcggcttgtaatacgactcactatagggcttttttttttttcggtttgagggggaatgctggagattgtaatgggtatggagacatatcatataagtaatgctagtcttatcctgtgtgaaattgttatccgct

SEQ ID NO: 59 - DNA, 730 bp, Homo sapien
aagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgaattgtaatacgactcactatagggcgaattgggccctctagatgcatgctcgagcggccgccagtgtgatggatatctgcagaattcggcttgtaatacgactcactatagggcttttttttttttcggtttgagggggaatgctggagattgtaatgggtatggagacatatcatataagtaatgctagtcttatcctgtgtgaaattgttatccgcta

SEQ ID NO: 60 - DNA, 623 bp, Homo sapien
gactccaagagaagactaggaagtagccctcgttctccagggcacccaaaataccagcctttattgtctgcatgattttaggggatatggggagggaacaagtagaagggaagagggaaatggagagcatccttatgactttacaaagggtggaaatgaggatggagggatacagaagtctgcacagctgtaaaggttttatagatgtctttgccttcccttctgaggaagggaagaagtaatgaaagcacatgtgaataaccccttccatcccattcacagcatcgcactcccagtccttaaggcaaagggaggcagtgctgaagcattggtggtgcagtgtaaagagacaagacctgatcatctgatcacacttgtgccaacttgattcatattgggcattactaacaacccctgggcaaggtaaataggttgaacaatcaataacattatccctgcctgcatacatgtgaacaaaagctatagaggacatgcaaattctacagtcattcctcatatgctttagacagagtgcagctactggaatcttccagatttcagtgctttaaaatcagagctctgaatacacaaaaaaaaaaagccctatagtgagtcgtattaca

SEQ ID NO: 61 - DNA, 376 bp, Homo sapien
gcatgctcgagcggccgccagtgtgatggatatctgcagaattcggcttagcggataacaatttcacacaggatccatgactcagctattaaggctctggccttggatccctatgaggaatattttaccacaggttcagcagaaggtaacataaaggtttggagattgacaggccatggcctaattcattcatttaaaagtgaacatgctaagcagtccatatttcgaaacattggggctggagtcatgcagattgacatcatccagggcaatcggctcttctcctgtggtgcagatggcacgctgaaaaccagggttttgcccaatgcttttaacatccctaacagaattcttgacattctataaagattggggt

SEQ ID NO: 62 - DNA, 539 bp, Homo sapien
atgactcattgtttctctgcctttccgtgtgttacaggtgggctgatccccctgcagccagtttcccataagcaactgacttccaactgggaatgtctcgggggataatgggggtggggatatggaagtatagagaaaacataagaaaatactgggtgtatacacctttctctctctgagtatgatgacaatgtgatagtcagtgtggcatctgcgactccagcttgtgcctggcatgtacaccctagctccagcttcccctgggagactgtgcatctcctggctccactaacaccaccttcttctgaccttccagcctagagatgatgactctgccagcctagatgggctctgggttgtctccctattcctgtttgctttgtagatttcccattatgctgtcaccaactccccagcctaagccctctctattttaaattctcaagtggattatgttcctgattagtccctgactgatataccactctcctcatgatctctgattagttttcctgttaggttgttgcagtaaaaaaaaa

SEQ ID NO: 63 - DNA, 304 bp, Homo sapien
ggcttagcggataacaatttcacacaggacgactccaagctgggaaggaaaattcccttttccaacctgtatcaatttttacaacttttttcctgaaagcagtttagtccatactttgcactgacatactttttccttctgtgctaaggtaaggtatccaccctcgatgcaatccaccttgtgttttcttagggtggaatgtgatgttcagcagcaaacttgcaacagactggccttctgtttgttactttcaaaaggcccacatgatacaattagagaattcccaccgcacaaaaaaaaaaag

SEQ ID NO: 64 - DNA, 226 bp, Homo sapien
  misc_feature (1)...(226): n = A,T,C or G
atgatgatgaccatgtggacagccaggactccattgactcgaacgactctgatgatgtngatgacactgatgattctcaccagtctgatgagtctcaccattctgatgaatctgatgaactggtcactgattttcccncggacctgccngcaaccgaagtnttcactccagttgtccccccagtagacacntntgatggccgaggtgatggtgtggtttatggact

SEQ ID NO: 65 - DNA, 225 bp, Homo sapien
  misc_feature (1)...(225): n = A,T,C or G
taccaacagagcttctgaaacagataccatagcattggagagaaaaacagctcacagtctgaggaagatgatattganagaaggaaagaattgaaagcatcttgaagaaaaactcagattggatntgggattggtcaagtcggccggataatattccccccaaggagttcctctttaaacacccgaagcgcacggccaccctcagcatgaggaacacgagcgtca

SEQ ID NO: 66 - DNA, 240 bp, Homo sapien
  misc_feature (1)...(240): n = A,T,C or G
ccagcatggtggccgtnatggatagcgacccacangcaagctgggctttgaggaattcaagtacttgtggaacaacatcaaaaggtggcaggccatatacaaacagtacgacactgaccgatcagggaccatgtgcagtagtgaactcccangtgcctttgaggcagcanggttccacctgaatgaacanctctataacatgatcatccgacnctactcagatgaaagtgggaacatgga

SEQ ID NO: 67 - DNA, 504 bp, Homo sapien
  misc_feature (1)...(504): n = A,T,C or G
cacgaggagagatngcatctgctatatattccacngatacatgtgagtnactgatagaaaaaatcgcnncggngaacactgncaccggtnccggcccccggtactacagggatctcntcagacttcaccgtntactacaangtaagcnccctttaagaatgtcacggagtatgatgggcaggatgcctgcggctccaacanctggaacntggtggacgtggacctcccgcccaacaaggacntggagcccggcatcttactacatgggctgaanccctggactcagtacgccgtttacntcaaggctgtgaccctcaccatggtggagaacgaccatatccgtggggccaagagtgagatcttgtncattcgcnccantgcttcngttccttccnttcccttggacnttctttcggcatcaaactcctcttctcagttaatcgtgaagtggaaccctccctctctgcccaacggcnacctgagttactactttgtgcnctggca

SEQ ID NO: 68 - DNA, 462 bp, Homo sapien
  misc_feature (1)...(462): n = A,T,C or G
tggatggcagggggagaaaggaaaagcaaaacactccaggacctctcccggatctgtctcctcctctagccagcagtatggacagctggacccctgaacttcctctcctcttacctgggcagagtgttgtctctccccaaatttataaaaactaaaatgcatnccattcctctgaaagcaaaacaaattcataattgagtgatattaaatanagaggttttcggaagcagatctgtgaatatgaaatacatgtgcatatttcattccccaggcagacattttttagaaatcaatacatgccccaatattggaaagacttgttcttccacggtgactacagtacatgctgaagcgtgccgtttcagccctcatttaattcaatttgtaagtagcgcagcagcctctgtgggggaggataggctgaaaaaaaaaaanccccttttttngtntnttttaaaaaaa

SEQ ID NO: 69 - DNA, 357 bp, Homo sapien
agaagtcttcctgagccttccatgtatcctcggtgcccggggattaaccagcgttatcaaccaaagctaaaggatgatgaggttgctcagctcaagaaaagtggagataccctgtgggacatccagaaggacctaaaagacctgtgactagtgagctctaggctgtagaaatttaaaaactacaatgtattaactcgatcctttagttttcatccatgtacatggatcacagtttgctttgatcttcttcaattgtgaatttgggctcacagaatcaaagcctatgcttggtttaatgcttgcaatctgagctcttgaacaaataaaattaactattgtagtgtgaaaaaaaaaaaa

SEQ ID NO: 70 - DNA, 226 bp, Homo sapien; identical to SEQ ID NO: 64
  misc_feature (1)...(226): n = A,T,C or G

SEQ ID NO: 71 - DNA, 477 bp, Homo sapien
  misc_feature (1)...(477): n = A,T,C or G
agcagacaagccacaattaacatagggtacaattgggtcatgtagctcatgggaaatccacagtcgtcaaagctatttctggagttcatactgtcaggttcaaaaatgaactagaaagaaatattacaatcaagcttggatatgctaatgctaagatttataagcttgatgacccaagttgccctcggccagaatgttatagatcttgtgggagcagtacacctgacgagtttcctacggacattccagggaccaaagggaacttcagattagtcagacatgtttcctttgttgactgtcctggccacnatattttgatggctactatgctgaacggtgcagcagtgatggatgcagctcttctgttgatagctggtaatgaatcttgccctcagcctcagacatcggaaacacctggctgctatagaagatcatgaaactggaagccatattttgaattctacaaaataaaattga

SEQ ID NO: 72 - DNA, 374 bp, Homo sapien
  misc_feature (1)...(374): n = A,T,C or G
ccaagccagattgtcactccagctgatcttctttgatggtgaagaggcttttcttcactggtctcctcaagattctctctatgggtctcgacacttaactgcaaagatggcatcgaccccgcacccacctggagcgagaggcaccagccaactgcatggcatggatttattggtcttattggatttgattggagctccaaacccaacgtttcccaatttttttccanactcagccaggtggttcgaangacttcaagcanttgaacatgaacttcatgaattgggtttgctcaangatcactctttggaggggcggtatttccanaattacagttatggaggtgtgattcaggatgaccnttttccatttccaa

SEQ ID NO: 73 - DNA, 597 bp, Homo sapien
  misc_feature (1)...(597): n = A,T,C or G
ccaagggatctgtaaagaatatatacttgagtggtgtgtgttatcagataaagcaccctgtatcacagactggcaacaagaagatggtaccgtgcatcgcacctatttaagagggaacttagcagagagcaaatgctatttgataacagttactccagtatatgctgatggaccaggaagccctgaatccataaaggcataccttaaacaagctccaccttccaaaggacctactgttcggacaaaaaaagtagggaaaaacgaagctgtcttanagtgggaccaacttcctgttgatgttcanaatggatttatcagaaattatactatattttatanaaccatcattggaaatgaaactgctgtgaatgtggattcttcccacacagaaatntacattgtcctctttgactagtgacacattgtacatggtacgaatggcagcatacacagatgaaggtgggaaggatggtccaaaattcacttttactaccccaaantttgctcaaggganaaattgaagccatantcgtgcctgtttgcttancattcctattgacaactcttctgggaatgctgttctgctttaataagcga

SEQ ID NO: 74 - DNA, 257 bp, Homo sapien
  misc_feature (1)...(257): n = A,T,C or G
tggtaaagggtaatagccagagnntagaaccttgangagatgcggccaangattctttatatctgaaccnagatgtnaaanaagaaaatgctttgaggctttctaagcgatcctcctgtctaatttncacctttgtctggatgcacacttctgaccncgctgccacaacctgtggggtctgatgtgtcccttgatgggtgcggccctcagggactgcaccctgacaagtgttnaggcaanattcctttcttgtgccc

SEQ ID NO: 75 - DNA, 330 bp, Homo sapien
  misc_feature (1)...(330): n = A,T,C or G
tgttcataaggctggtgatanaggggtcttgtcatggaaaggtgctcttccaggaaacctctgtgtatggaggtcgnagccacaatacgcggacgangatgtgaacacctacaatgccgccatcncttacaccatcctcagccaagatcctgagctccctgacnaaaatatgttcnccattaacaggaacgcaggagtcatcggtgtggtcnccactgggctggaccgaaagagtttccctacgtgtaccntggtggttcaagcngctgaccttcanggtgaggggttaatcacnacagcancngctgtgatcacagtcactgntaccaa

SEQ ID NO: 76 - DNA, 387 bp, Homo sapien
  misc_feature (1)...(387): n = A,T,C or G
gctcgcgcgcctgcaggtcgacactagtggatccaaagaattcggcacgagaacaacagttatctccaagatgctattcgttgaacccatcctggaggtttccagcttgccgacaaccaactcaacaaccaattcagccaccaaaataacagctaataccactgatgaacccaccacacaacccaccacagagcccaccacccaacccaccatccaacccacccaaccaactacccagctcccaacagattctcctacccagcccactactgggtccttctgcccaggacctgttactctctgctctgacttgganantcattcaacanaagccgtgttgggggaagctttggtaaatttctccctgaagctctaccacgccttctc

SEQ ID NO: 77 - DNA, 339 bp, Homo sapien
  misc_feature (1)...(339): n = A,T,C or G
ctgctgatcngggtccctttggagcacagatgatgcnatggccancnngggacaacnacgtgatctgcgccctggtcctggtgtccatnctggccctcggnancctggccgaggcccanacanagacgtgtncagtggccccccgtgaaagacagaattgtggttttcctggtgtcacaccctcccantgtgcaaataagggctgctgtttcgacaacaccgttcgtggggtcccctggtgcttctatcctaataccntcnacntcccnccanaaaaggantgtgaattttanacacttctgcagggatctgcctgcatcctgacgcngtgccgtcccc

SEQ ID NO: 78 - DNA, 385 bp, Homo sapien
tcggtcatagggagagatttgtatgctgtactatgcagcgtttaaagttagtgggttttgtgatttttgtattgaatattgctgtctgttacaaagtcagttaaaggtacgttttaatatttaagttattctatcttggagataaaatctgtatgtgcaattcaccggtattaccagtttattatgtaaacaagagatttggcatgacatgttctgtatgtttcagggaaaaatgtctttaatgctttttcaagaactaacacagttattcctatactggattttaggtctctgaagaactgctggtgtttaggaataagaatgtgcatgaagcctaaaataccaagaaagcttatactgaatttaagcaaaaaaaaaaaacccc

SEQ ID NO: 79 - DNA, 307 bp, Homo sapien
  misc_feature (1)...(307): n = A,T,C or G
tcgatacagggatgtcagagctgccagagactttatcctgaagctttaccaagatcagaatcctgacaaagnagaaagtcatctactctcacttcacatgtgctacagatacagacaatattcgctttgtgtttgctgctgtcaaagacacaattctacagctaancctaagggaattcaaccttgtctaaaagctgctgcccactcctcccctataacagaagatgtgatttgcaaactccttgttttatttgnaagtgcttctgacatcnccagagccagccccatgccaggaactaaggatgtc

SEQ ID NO: 80 - DNA, 528 bp, Homo sapien
  misc_feature (1)...(528): n = A,T,C or G
gtcgatacaggaacagcatgtccaaatcgatgtggatgtttccaagcctgacctcacggctgccctgcgtgacgtacgtcagcaatatgaaagtgtggctgccaagaacctgcaggaggcagaagaatggtacaaatccaagtttgctgacctctctgaggctgccaaccggaacaatgacgccctgcgccaggcaaagcaggagtccactgagtaccggagacaggtgcagtccctcacctgtgaagtggatgcccttaaaggaaccaatgagtccctggaacgccagatgcgttgaaatggaagagaactttgccgttgaagctgctaactaccaagacactattggccgcctgcaggatgagattcagaatatgaagganggaaatggctcgtcaccttcgtgaataccaagacctgctcaatgttaagatggcccttgacattgaaattgccacctacanggaactgctggangcnaagaaaaccaggatttctctgcctcctccnaacttttcctcccctgaa

SEQ ID NO: 81 - DNA, 369 bp, Homo sapien
agcatggctcccgaagttttgccaaaacctcggatgcgtggccttctggccaggcgtctgcgaaatcatatggctgtagcattcgtgctatccctgggggttgcagctttgtataagtttcgtgtggctgatcaaagaaagaaggcatacgcagatttctacagaaactacgatgtcatgaaagattttgaggagatgaggaaggctggtatctttcagagtgtaaagtaatcttggaatataaagaatttcttcaggttgaattacctagaagtttgtcactgacttgtgttcctgaactatgacacatgaatatgtgggctaagaaatagttcctcttgataaataaacaattaacaaaaaaaaaaa

SEQ ID NO: 82 - DNA, 269 bp, Homo sapien
  misc_feature (1)...(269): n = A,T,C or G
atgacagggatgancaaacttngtctggggtattgatgaagatgacctactgctgatgataccagtgctgctgtaactgaagaaatgccaccccttgaaggagatgacgacacatcacgcatggaagaagtagactaatctctggctgagggatgacttacctgttcagtactctacaattcctctgataatatattttcaaggatgtttttctttatttttgttaatattaaaangtctgtntggnatgacaactnctttaaggggaa

SEQ ID NO: 83 - DNA, 196 bp, Homo sapien
  misc_feature (1)...(196): n = A,T,C or G
tttgggtccaattacagctaaagcaaaagtggttattgaactgtttttatcggtctcgggnnttgctaaaccttcccaggtgtattttggaggtacagttgttggcnagcaagctatnaaatctgaagatgaagtgggaagttnaatanagtatgaatncagggtaagaaactnaggtaaacctcnaatatncctc

SEQ ID NO: 84 - DNA, 448 bp, Homo sapien
  misc_feature (1)...(448): n = A,T,C or G
caaacatgggcatggtgtcagcgataatgtttntancagctcccgacataaatcagtaantnngatttccaccatatcnancntcnggaatttaaccntcaggagnagctcttnntcagacnccctggaaaaacgagccccattgnanccanctttganacataaaacctggagaaattctccaatacngaaggtatanagcggggcatcgttgacagcatcacgggtcaaaggcttctggaggctcaggcctgcaaaggtggcatcatccacccaaccacgggccagaacctgtcncttcaggacgcagtctcccngggtgtgattgaccaagacatggccaccaggctgaagcctgctcagaaagccttcataggcttcgagggtgtgaagggaaagaagaagatgtcagcagcagaggcagtgaaaaaaaaaaaacccctatatt

SEQ ID NO: 85 - DNA, 169 bp, Homo sapien
agcagaccaactgccttttgtgagaccttcccctccctatccccaactttaaaggtgtgagagtattaggaaacatgagcagcatatggcttttgatcagtttttcagtggcagcatccaatgaacaagatcctacaagctgtgcaggcaaaacctagcaggaaaaaaa